(19) United States
(12) Patent Application Publication
(10) Pub. No.: US 2013/0040998 A1
(43) Pub. Date: Feb. 14, 2013

(54) FLUORINATED HDAC INHIBITORS AND USES THEREOF

(71) Applicants: James Elliot Bradner, Cambridge, MA (US); Ralph Mazitschek, Belmont, MA (US)

(72) Inventors: James Elliot Bradner, Cambridge, MA (US); Ralph Mazitschek, Belmont, MA (US)

(73) Assignees: Dana-Farber Cancer Institute, Inc., Boston, MA (US); President and Fellows of Harvard College, Cambridge, MA (US)

(21) Appl. No.: 13/520,650

(22) PCT Filed: Jan. 5, 2011
(86) PCT No.: PCT/US2011/020206

§ 371 Date: Oct. 17, 2012

Related U.S. Application Data
(60) Provisional application No. 61/293,338, filed on Jan. 8, 2010.

(51) Int. Cl. A61K 31/165; A61K 31/4045; C07C 239/14; A61P 35/00; A61K 31/40; C07D 213/56; A61K 31/44; C07D 209/14; C07D 207/333

(52) U.S. Cl. 514/355; 548/507; 514/415; 564/182; 514/617; 548/540; 514/423; 546/316

(57) ABSTRACT

Fluorinated deacetylase inhibitors of the general formulae (I), (II), and (III): and pharmaceutically acceptable salts thereof, as described herein, are useful as inhibitors of histone deacetylases or other deacetylases, and thus are useful for the treatment of various diseases and disorders associated with acetylase activity as described herein (e.g., cancer, neurodegenerative diseases, inflammatory diseases).

### RELATED APPLICATIONS

The present application claims priority under 35 U.S.C. §119(e) to U.S. provisional patent application, U.S. Ser. No. 61/293,338, filed Jan. 8, 2010, which is incorporated herein by reference.

### GOVERNMENT SUPPORT

This invention was made with U.S. Government support under grants CA078048 and CA128972 awarded by the National Institutes of Health. The U.S. Government has certain rights in the invention.

### BACKGROUND OF THE INVENTION

A biological target of recent interest in the identification of small organic molecules as therapeutic agents is histone deacetylase (see, for example, a discussion of the use of inhibitors of histone deacetylases in the treatment of cancer: Marks et al. Nature Reviews Cancer 2001, 1, 194; Johnstone et al. Nature Reviews Drug Discovery 2002, 1, 287). Post-translational modification of proteins (e.g., histones, transcription factors, tubulin) through the acetylation and deacetylation of lysine residues has a critical role in regulating their biological function. HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). The function of other proteins such as tubulin is also thought to be regulated by their acetylation state. HDACs participate in cellular pathways that control cell shape and differentiation, and an HDAC inhibitor has been shown effective in treating an otherwise recalcitrant cancer (Warrell et al. J. Natl. Cancer Inst. 1998, 90, 1621-1625). Eleven human HDACs, which use zinc as a cofactor, have been characterized (Taunton et al. Science 1996, 272, 408-411; Yang et al. J. Biol. Chem. 1997, 272, 28001-28007; Grozinger et al. Proc. Natl. Acad. Scl. U.S.A. 1999, 96, 4868-4873; Kao et al. Genes Dev. 2000, 14, 55-66; Hu et al. J. Biol. Chem. 2000, 275, 15254-15264; Mon et al. Proc. Natl. Acad. Sci. U.S.A. 2001, 98, 10572-10577; Venter et al. Science 2001, 291, 1304-1351). These members fall into three related classes (Class I, II, and IV) (Gregoretti et al., J. Mol. Biol. 2004, 338, 17-31). Class I HDACs include HDAC1, HDAC2, and HDAC3. Class II includes HDAC4, HDAC5, HDAC6, HDAC7, HADC9, and HDAC10. Class II is further subdivided into Class IIa, which includes HDAC4, HDAC5, HDAC7, and HDAC9, and Class IIb, which includes HDAC6 and HDAC10. Class IV includes HDAC11. An additional Class of HDACs has been identified which use NAD as a cofactor. These have been termed Class III deacetylases, also known as the sirtuin deacetylases. Based on this understanding of known HDACs and other deacetylases in the cells, efforts are currently focused on developing novel deacetylase inhibitors

### SUMMARY OF THE INVENTION

The present invention provides novel fluorinated deacetylase inhibitors and methods of preparing and using these compounds. These novel deacetylase inhibitors (e.g., histone deacetylase (HDAC), tubulin deacetylase (TDAC)) are useful as research tools as well as for the treatment of various deacetylase-associated diseases, including, but not limited to, proliferative diseases, such as cancer; autoimmune diseases; allergic and inflammatory diseases; diseases of the central nervous system (CNS), such as neurodegenerative diseases (e.g., Huntington's disease); vascular diseases, such as restenosis; musculoskeletal diseases; cardiovascular diseases, such stroke and myocardial infarction; pulmonary diseases; genetic diseases; infectious diseases; and gastric diseases. The present invention stems at least in part from the discovery that the fluorination of hydroxamic acid-based deacetylase inhibitors results in an increase in acidity of the hydroxamic acid moiety. This increase in acidity renders the compounds more reactive with deacetylases.

In one aspect, the present invention provides novel fluorinated compounds useful for inhibition of deacetylases. In certain embodiments, a deacetylase inhibitor of the invention can be represented by the formula A-B—C, in which A is a specificity element for selective binding to a deacetylase, B is a fluorinated linker element, and C is a chelator moiety (e.g., a hydroxamic acid moiety). In one embodiment, there is provided a composition for inhibiting a deacetylase comprising a compound represented by the general formula A-B—C, wherein
A is selected from the group consisting of cycloalkyls, unsubstituted and substituted aryls, heterocyclyls, amino aryls, and cyclopeptides;
B includes at least one fluorine and is selected from the group consisting of substituted or unsubstituted C₄-C₈ alkylidenes, C₄-C₈ alkenylidenes, C₄-C₈ alkynylidenes, and -(D-E-F)—, in which D and F are, independently, absent or represent a C₂-C₇ alkylidene, a C₂-C₇ alkenylidene, or a C₂-C₇ alkynylidene, and E represents O, S, or NR′, in which R′ represents H, a lower alkyl, a lower alkenyl, a lower alkynyl, an aralkyl, aryl, or a heterocyclyl; and
C is selected from the group consisting of:

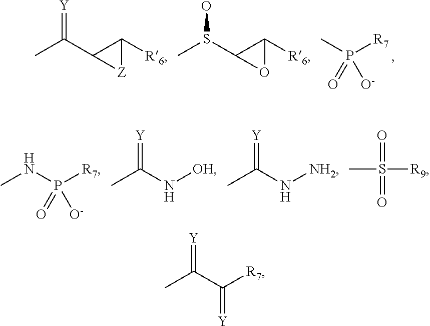

and boronic acid;
in which
Z represents O, S, or NR;
Y represents O or S;
R₅ represents a hydrogen, an alkyl, an alkoxycarbonyl, an aryloxycarbonyl, an alkylsulfonyl, an arylsulfonyl, or an aryl;
R′₆ represents hydrogen, an alkyl, an alkenyl, an alkynyl or an aryl;
R₇ represents a hydrogen, an alkyl, an aryl, an alkoxy, an aryloxy, an amino, a hydroxylamino, an alkoxylamino or a halogen; and
R₉ represents a hydrogen, an alkyl, an aryl, a hydroxyl, an alkoxy, an aryloxy, or an amino.

In certain embodiments, the novel fluorinated compounds are of general formula (I), (II), or (III):

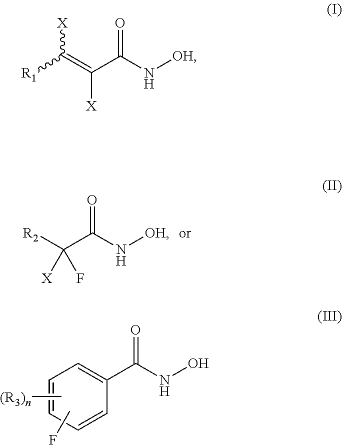

and pharmaceutically acceptable salts thereof, as described herein. The compounds are useful as inhibitors of histone deacetylases or other deacetylases (e.g., tubulin deacetylase), and thus are useful for the treatment of various diseases and disorders associated with deacetylase activity as described herein. The inventive compounds are additionally useful as tools to probe biological function. Exemplary inventive deacetylase inhibitors with a 2-fluoro-N-hydroxy-acrylamide include compounds of the formulae:

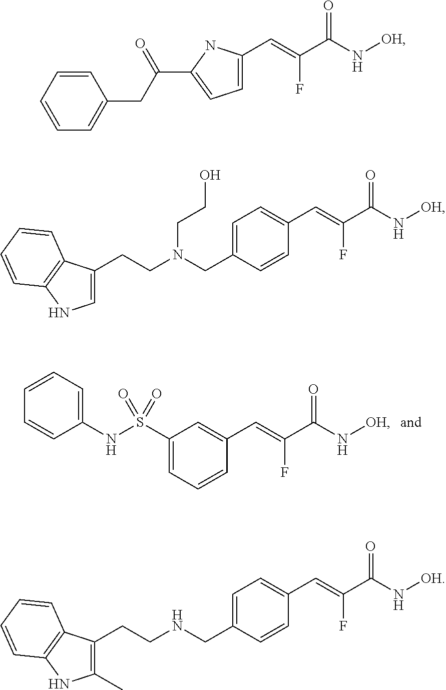 [SMILES: O=C(NO)/C(F)=C/c1ccc(C(=O)Cc2ccccc2)[nH]1 ; O=C(NO)/C(F)=C/c1ccc(CN(CCO)CCc2c[nH]c3ccccc23)cc1 ; O=C(NO)/C(F)=C/c1cccc(S(=O)(=O)Nc2ccccc2)c1 ; Cc1[nH]c2ccccc2c1CCNCc1ccc(/C=C(\F)C(=O)NO)cc1]

Another exemplary inventive deacetylase inhibitor with a 2-fluoro-N-hydroxy-acrylamide is of the formula:

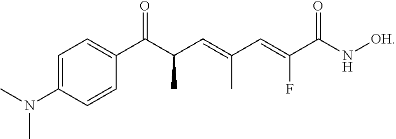 [SMILES: CC(/C=C(\F)C(=O)NO)=C\[C@@H](C)C(=O)c1ccc(N(C)C)cc1]

Other exemplary inventive deacetylase inhibitors with a 2-fluoro-N-hydroxy-alkylamide include compounds of the formulae:

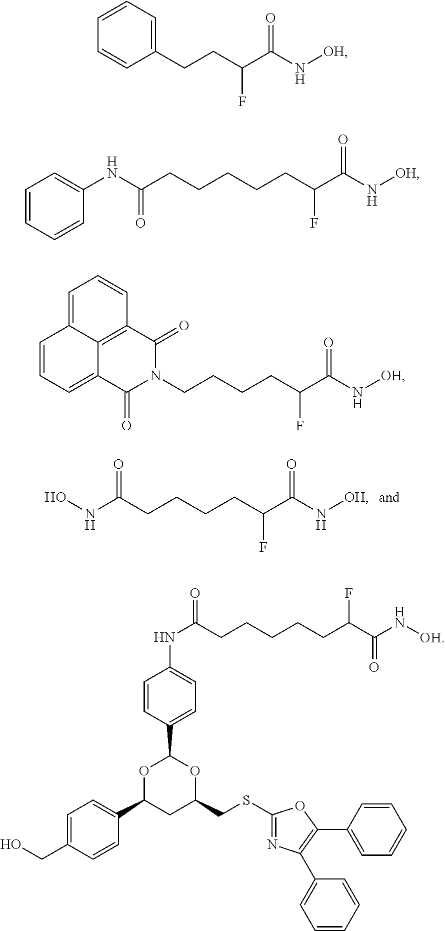

Exemplary inventive deacetylase inhibitors with a fluorinated N-hydroxy-benzamide include compounds of the formulae:

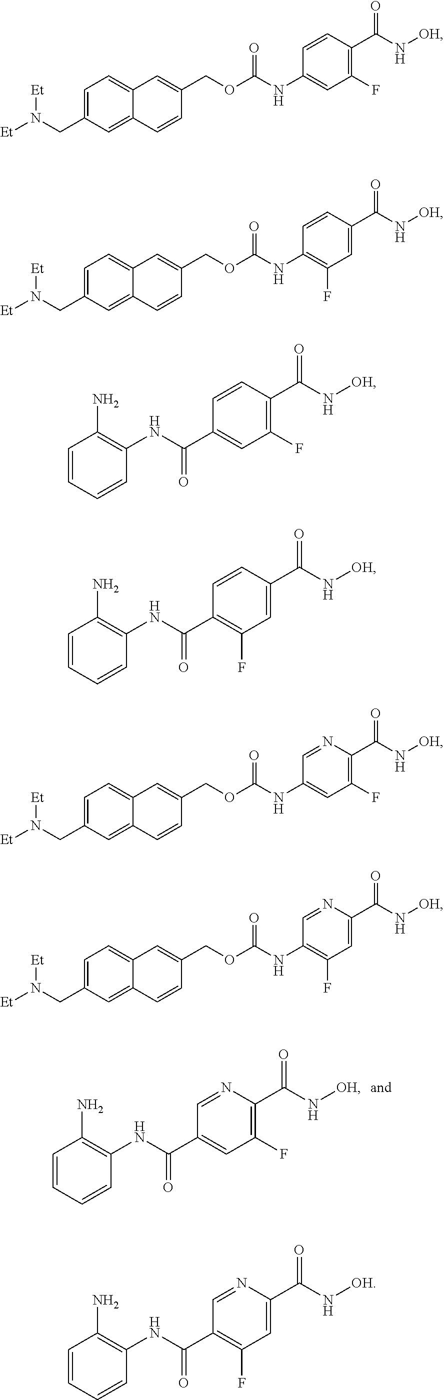

Other exemplary fluorinated deacetylase inhibitors include compounds of the formulae:

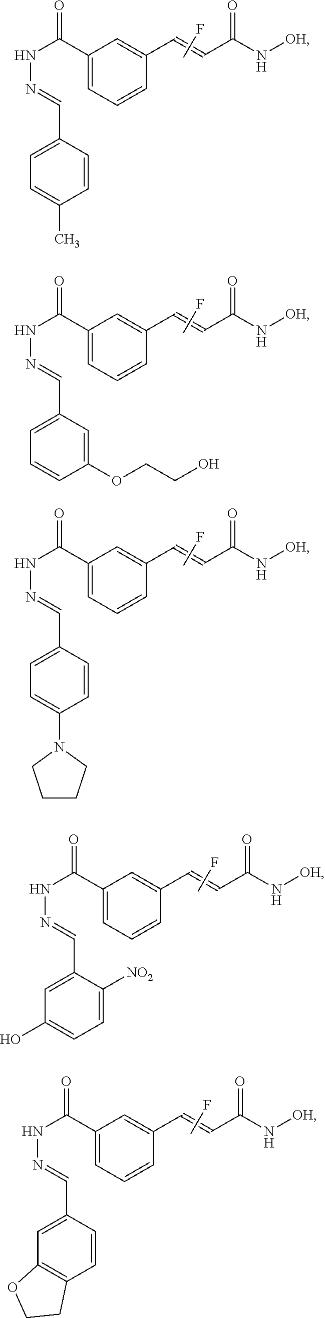

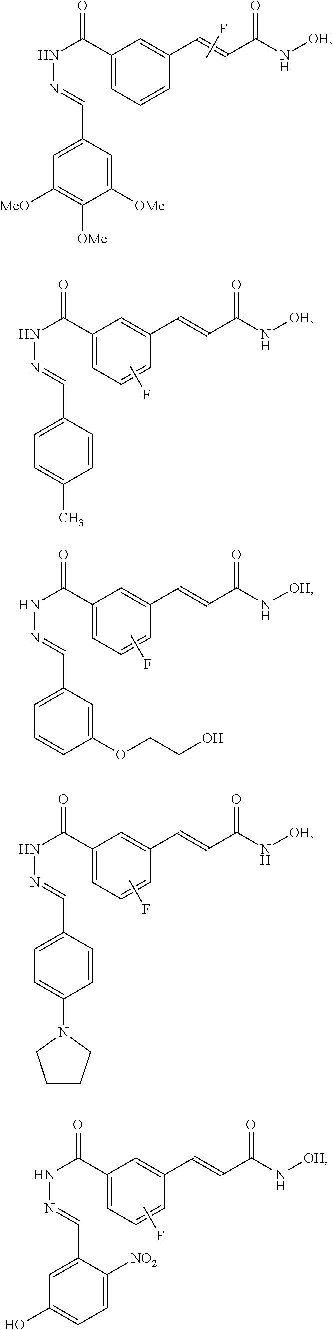

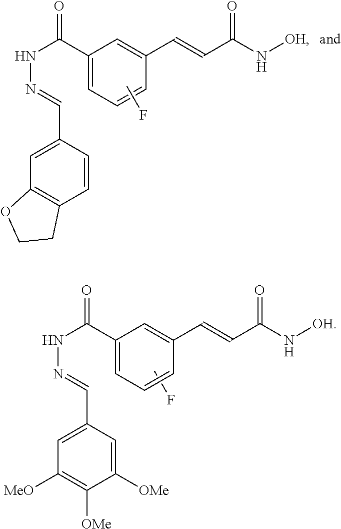

In another aspect, the present invention provides methods for inhibiting histone deacetylase activity or other deacetylase activity in a subject or a biological sample, comprising administering to said subject, or contacting said biological sample, with an effective inhibitory amount of a compound of the invention. In certain embodiments, the compound specifically inhibits a particular HDAC isoform (e.g., HDAC1, HDAC2, HDAC3, HDAC4, HDAC5, HDAC6, HDAC7, HDAC8, HDAC9, HDAC10, HDAC11) or class of HDACs (e.g., Class I, II, or IV). In still another aspect, the present invention provides methods for treating diseases or disorders involving histone deacetylase activity, comprising administering to a subject in need thereof a therapeutically effective amount of a compound of the invention. In certain embodiments, the disease can be proliferative diseases, such as cancer; autoimmune diseases; allergic and inflammatory diseases; diseases of the central nervous system (CNS), such as neurodegenerative diseases (e.g., Huntington's disease, amyotrophic lateral sclerosis (ALS)); vascular diseases, such as restenosis; musculoskeletal diseases; cardiovascular diseases, such as stroke; pulmonary diseases; gastric diseases; genetic diseases, such as spinal muscle atrophy; infectious diseases; diseases associated with an HPV infection; and Alzheimer's disease. The compounds may be administered to a subject by any method known in the art. In certain embodiments, the compounds are administered paranterally or orally. The compounds may also be administered topically. The invention also provides pharmaceutical compositions comprising a therapeutically effective amount of an inventive compounds and optionally a pharmaceutically acceptable excipient.

In another aspect, the present invention provides methods of preparing the inventive fluorinated deacetylase inhibitors as described herein. In certain embodiments, the inventive compounds are prepared based on syntheses of the non-fluorinated compounds known in the art.

In certain other aspects, the present invention provides a kit comprising at least one container having an inventive compound or pharmaceutical composition thereof, and instructions for use. In other aspect of the invention the container comprises multiple dosage units of an inventive pharmaceutical composition. For example, the kit may include a whole treatment regimen of the inventive compound (e.g., a week long course, a round of chemotherapy).

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Furthermore, it will be appreciated by one of ordinary skill in the art that the synthetic methods, as described herein, utilize a variety of protecting groups. By the term “protecting group,” has used herein, it is meant that a particular functional moiety, e.g., C, O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference. Furthermore, a variety of carbon protecting groups are described in Myers, A.; Kung, D. W.; Zhong, B.; Movassaghi, M.; Kwon, S. J. Am. Chem. Soc. 1999, 121, 8401-8402, the entire contents of which are hereby incorporated by reference.

An “O-protecting group”, as described herein, refers to any hydroxyl protecting group known to one of ordinary skill in the art. Such protecting groups include but are not limited to ethers, such as substituted alkyl ethers, substituted methyl ethers, substituted ethyl ethers, substituted benzyl ethers, silyl ethers, as well as esters, carbonates, and sulfonates.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of HDAC-associated diseases (e.g., cancer). The term “stable”, as used herein, preferably refers to compounds which possess stability sufficient to allow manufacture and which maintain the integrity of the compound for a sufficient period of time to be detected and preferably for a sufficient period of time to be useful for the purposes described herein.

The term “acyl”, as used herein, refers to a carbonyl-containing functionality, e.g., —C(═O)R, wherein R is an aliphatic, alycyclic, heteroaliphatic, heterocyclic, aryl, heteroaryl, (aliphatic)aryl, (heteroaliphatic)aryl, heteroaliphatic(aryl), or heteroaliphatic(heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality (e.g., forming a carboxylic acid, ester, or amide functionality).

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, and alkynyl moieties. Thus, as used herein, the term “alkyl” includes straight and branched alkyl groups. An analogous convention applies to other generic terms such as “alkenyl”, “alkynyl”, and the like.

Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 14 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties, and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alicyclic” or “carbocyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclopentyl, cyclopentyl, —CH₂-cyclopentyl, cyclohexyl, —CH₂-cyclohexyl, cyclohexenylethyl, cyclohexanylethyl, norbornyl moieties, and the like, which may bear one or more substituents.

The term “alkoxy” or “alkyloxyl” or “thioalkyl”, as used herein, refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include, but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl contains 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino, n-propylamino, and the like.

Some examples of substituents of the above-described aliphatic (and other) moieties of compounds of the invention include, but are not limited to, aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The term “alkylidene,” as used herein, refers to a substituted or unsubstituted, linear or branched saturated divalent radical consisting solely of carbon and hydrogen atoms, having from one to n carbon atoms, having a free valence “-” at both ends of the radical. In certain embodiments, the alkylidene moiety has 1 to 6 carbon atoms. The term “alkenylidene”, as used herein, refers to a substituted or unsubstituted, linear or branched unsaturated divalent radical consisting solely of carbon and hydrogen atoms, having from two to n carbon atoms, having a free valence “-” at both ends of the radical, and wherein the unsaturation is present only as double bonds and wherein a double bond can exist between the first carbon of the chain and the rest of the molecule. In certain embodiments, the alkenylidene moiety has 2 to 6 carbon atoms.

The term “alkynylidene,” as used herein, refers to a substituted or unsubstituted, linear or branched unsaturated divalent radical consisting solely of carbon and hydrogen atoms, having from two to n carbon atoms, having a free valence “-” at both ends of the radical, and wherein the unsaturation is present only as triple or doulbe bonds and wherein a triple or double bond can exist between the first carbon of the chain and the rest of the molecule. In certain embodiments, the alkynylidene moiety has 2 to 6 carbon atoms.

Unless otherwise indicated, as used herein, the terms “alkyl”, “alkenyl”, “alkynyl”, “heteroalkyl”, “heteroalkenyl”, “heteroalkynyl”, “alkylidene”, alkenylidene”, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl” and the like encompass both substituted and unsubstituted groups.

The term “amino”, as used herein, refers to a primary (—NH₂), secondary (—NHRₓ), tertiary (—NRₓR_{y}), or quaternary (—N⁺RₓR_{y}R₂)amine, where Rₓ, R_{y} and R_{z} are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

In general, the term “aromatic moiety,” as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2), wherein n is an integer. A mono- or polycyclic, unsaturated moiety that does not satisfy one or all of these criteria for aromaticity is defined herein as “non-aromatic,” and is encompassed by the term “alicyclic.”

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S, and N within the ring (i.e., in place of a ring carbon atom). In certain embodiments, the term “heteroaromatic moiety” refers to a planar ring comprising at least on heteroatom, having p-orbitals perpendicular to the plane of the ring at each ring atom, and satisfying the Huckel rule where the number of pi electrons in the ring is (4n+2), wherein n is an integer. It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like.

The term “heteroaryl”, as used herein, does not differ significantly from the common meaning of the term in the art, and refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one or more of the hydrogen atoms thereon independently with any one or more of the following moieties including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; and —NRₓ(CO)Rₓ; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or (alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl, and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments described herein.

The term “heteroaliphatic,” as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to, aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; and —NRₓ(CO)Rₓ; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described herein may be substituted or unsubstituted. Additional examples of generally applicable substituents are illustrated by the specific embodiments described herein.

The term “heterocycloalkyl,” “heterocycle,” or “heterocyclic,” as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S, and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6-, or 7-membered ring or a polycyclic group wherein at least one ring atom is a heteroatom selected from O, S, and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroaryloxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, but is not limited to, aliphatic, alicyclic; heteroaliphatic, heterocyclic, aromatic, heteroaromatic, aryl, heteroaryl, alkylaryl, alkylheteroaryl, heteroalkylaryl, or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, or heteroaryl substituents described herein may be substituted or unsubstituted. Additional examples or generally applicable substituents are illustrated by the specific embodiments described herein.

Additionally, it will be appreciated that any of the alicyclic or heterocyclic moieties described herein may comprise an aryl or heteroaryl moiety fused thereto. Additional examples of generally applicable substituents are illustrated by the specific embodiments described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like. In certain embodiments, the alkyl group is perhalogenated (e.g., perfluorinated).

The term “amino,” as used herein, refers to a primary (—NH₂), secondary (—NHRₓ), tertiary (—NRₓR_{y}), or quaternary (—N⁺RₓR_{y}R_{z})amine, where Rₓ, R_{y}, and R_{z}, are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “alkylidene,” as used herein, refers to a substituted or unsubstituted, linear or branched saturated divalent radical of carbon and hydrogen atoms, having from one to n carbon atoms and having a free valence at both ends of the radical. The alkylidene moiety may be substituted.

The term “alkenylidene”, as used herein, refers to a substituted or unsubstituted, linear or branched unsaturated divalent radical of carbon and hydrogen atoms, having from two to n carbon atoms and having a free valence at both ends of the radical, and wherein the unsaturation is present only as double bonds and wherein a double bond can exist between the first carbon of the chain and the rest of the molecule. The alkenylidene moiety may be substituted.

The term “alkynylidene”, as used herein, refers to a substituted or unsubstituted, linear or branched unsaturated divalent radical of carbon and hydrogen atoms, having from two to n carbon atoms, having a free valence“−” at both ends of the radical, and wherein the unsaturation is present only as triple bonds and wherein a triple bond can exist between the first carbon of the chain and the rest of the molecule. The alkynylidene moiety may be substituted.

The term “carbamate”, as used herein, refers to any carbamate derivative known to one of ordinary skill in the art. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, N-(2-pivaloylamino)-1,1-dimethylethyl carbamate, NpSSPeoc. In certain embodiments, carbamates are used as nitrogen protecting groups.

Unless otherwise indicated, as used herein, the terms “alkyl”, “alkenyl”, “alkynyl”, “heteroalkyl”, “heteroalkenyl”, “heteroalkynyl”, “alkylidene”, “alkynylidene”, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl”, and the like encompass both substituted and unsubstituted groups.

The phrase, “pharmaceutically acceptable derivative,” as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. The biological activity of pro-drugs may also be altered by appending a functionality onto the compound, which may be catalyzed by an enzyme. Also, included are oxidation and reduction reactions, including enzyme-catalyzed oxidation and reduction reactions. Certain exemplary pharmaceutical compositions and pharmaceutically acceptable derivatives are discussed in more detail herein.

“Compound”: The term “compound” or “chemical compound” as used herein can include organometallic compounds, organic compounds, transitional metal complexes, and small molecules. In certain embodiments, polynucleotides are excluded from the definition of compounds. In other embodiments, polynucleotides and peptides are excluded from the definition of compounds. In certain embodiments, the term compound refers to small molecules (e.g., preferably, non-peptidic and non-oligomeric) and excludes peptides, polynucleotides, transition metal complexes, metals, and organometallic compounds.

“Small Molecule”: As used herein, the term “small molecule” refers to a non-peptidic, non-oligomeric organic compound, either synthesized in the laboratory or found in nature. A small molecule is typically characterized in that it contains several carbon-carbon bonds, and has a molecular weight of less than 2000 g/mol, preferably less than 1500 g/mol, although this characterization is not intended to be limiting for the purposes of the present invention. Examples of “small molecules” that occur in nature include, but are not limited to, taxol, dynemicity and rapamycin. Examples of “small molecules” that are synthesized in the laboratory include, but are not limited to, compounds described in Tan et al. (“Stereoselective Synthesis of over Two Million Compounds Having Structural Features Both Reminiscent of Natural Products and Compatible with Miniaturized Cell-Based Assays” J. Am. Chem. Soc. 1998, 120, 8565; incorporated herein by reference).

“HDAC”: The term “HDAC” or “HDACs” refers to histone deacetylase(s).

“TDAC”: The term “TDAC” or “TDACs” refers to tubulin deacetylase(s).

“Deacetylase activity”: The term “deacetylase activity” refers to the regulation of a cellular process by modulating protein structure and/or function by the removal of an acetyl group.

“Biological sample”: As used herein the term “biological sample” includes, without limitation, cell cultures, or extracts thereof; biopsied material obtained from an animal (e.g., mammal) or extracts thereof; and blood, saliva, urine, feces, semen, tears, or other body fluids or extracts thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g., blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g., fluid obtained from an abscess or any other site of infection or inflammation), or fluid obtained from a joint (e.g., a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue, or organ. Biological samples may also include sections of tissues such as frozen sections taken for histological purposes. Biological samples also include mixtures of biological molecules including proteins, lipids, carbohydrates, and nucleic acids generated by partial or complete fractionation of cell or tissue homogenates. Although the sample is preferably taken from a human subject, biological samples may be from any animal, plant, bacteria, virus, yeast, etc.

“Animal”: The term animal, as used herein, refers to humans as well as non-human animals, at any stage of development, including, for example, mammals, birds, reptiles, amphibians, fish, worms, and single cells. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a clone.

“Pharmaceutically acceptable salt”: As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of amines, carboxylic acids, and other types of compounds, are well known in the art. For example, Berge et al. describe pharmaceutically acceptable salts in detail in J. Pharmaceutical Sciences 1977, 6, 1-19, incorporated herein by reference. The salts can be prepared in situ during the final isolation and purification of a compound of the invention, or separately by reacting a free base or free acid function with a suitable reagent, as described generally below. For example, a free base can be reacted with a suitable acid. Furthermore, where the compound of the invention carries an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### BRIEF DESCRIPTION OF THE DRAWINGS

FIG. 1 illustrates the chemical structures of exemplary deacetylase inhibitor, which can be fluorinated based on the present invention.

FIG. 2 demonstrates that the more acidic fluorohydroxamic acid MAZ1702 exhibits significantly increased affinity for class IIa HDAC enzymes compared to the less acidic analog MAZ1704.

FIG. 3 illustrates a synthesis of α-fluoro cinnamic hydroxamic acids and it use in the synthesis of fluorinated analogs of LBH-589 (e.g., LBF).

FIG. 4 shows the results of profiling a fluorinated analog of LBH-589 (LBF) against human HDAC1-HDAC9.

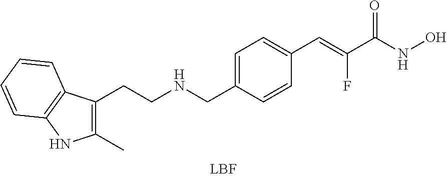 [SMILES: Cc1[nH]c2ccccc2c1CCNCc1ccc(/C=C(\F)C(=O)NO)cc1]

FIG. 5 illustrates a synthetic strategy for preparing α,β-difluoro cinnamic hydroxamates.

FIGS. 6A and 6B illustrates the inhibitory activity (IC₅₀ determination) of LBH-589 against HDACs1-9.

FIGS. 7A and 7B illustrates the inhibitory activity (IC₅₀ determination) of LBF against HDACs1-9.

FIG. 8 illustrates the inhibitory activity (IC₅₀ determination) of MAZ1702 against HDAC4, HDAC5, HDAC7, HDAC8, and HDAC9.

FIG. 9 illustrates the inhibitory activity (IC₅₀ determination) of MAZ1704 against HDAC4, HDAC5, HDAC7, HDAC8, and HDAC9.

### DETAILED DESCRIPTION OF CERTAIN EMBODIMENTS OF THE INVENTION

As discussed above, there remains a need for the development of novel deacetylase inhibitors. The present invention provides novel compounds of the general formulae A-B—C, (I), (II), and (III) and methods for the synthesis thereof, which compounds are useful as inhibitors of deacetylases (e.g., histone deacetylases), and thus are useful for the treatment of diseases or disorders associated with deacetylase activity. In certain embodiments, the inventive compounds are useful in the treatment of proliferative diseases, such as cancer; autoimmune diseases; allergic and inflammatory diseases; diseases of the central nervous system (CNS), such as neurodegenerative diseases (e.g. Huntington's disease); vascular diseases, such as restenosis; musculoskeletal diseases; cardiovascular diseases, such as stroke; pulmonary diseases; and gastric diseases. In particular, the inventive compounds are cinnamic hydroxymates. In certain embodiments, the compounds are class-specific. In certain embodiments, the compounds are isoform-specific. In other embodiments, the compounds are class I HDAC inhibitors. In certain embodiments, the compounds of the invention are class IIa HDAC inhibitors. In still other embodiments, the compounds are class IIb HDAC inhibitors. In certain embodiments, the compounds are class III HDAC inhibitors. In certain embodiments, the compounds are class IV HDAC inhibitors.

### Compounds of the Invention

Compounds of this invention include those, as set forth above and described herein, and are illustrated in part by the various classes, subclasses, subgenera, and species disclosed herein.

In certain embodiments, the present invention provides compounds for inhibiting a deacetylase of the general formula A-B—C, wherein:
A is selected from the group consisting of cycloalkyls, unsubstimted and substituted aryls, heterocyclyls, amino aryls, and cyclopeptides;
B includes at least one fluorine and is selected from the group consisting of substituted C₄-C₈ alkylidenes, C₄-C₈ alkenylidenes, C₄-C₈ alkynylidenes, and -D-E-F)—, in which D and F are, independently, absent or represent a C₂-C₇ alkylidene, a C₂-C₇ alkenylidene or a C₂-C₇ alkynylidene; and E represents O, S, or NR′; in which R′ represents H, lower alkyl, lower alkenyl, lower alkynyl, aralkyl, aryl, or heterocyclyl; and
C is selected from the group consisting of:

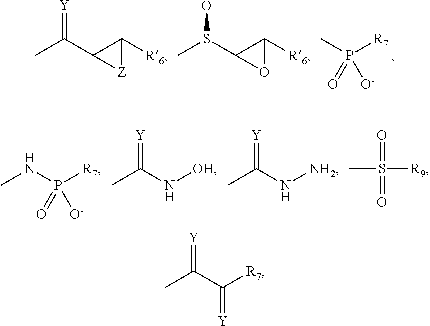

and boronic acid;
wherein
Z represents O, S, or NR;
Y represents O or S;
R₅ represents hydrogen, alkyl, alkoxycarbonyl, aryloxycarbonyl, alkylsulfonyl, arylsulfonyl, or aryl;
R′₆ represents hydrogen, an alkyl, an alkenyl, an alkynyl, or an aryl;
R₇ represents a hydrogen, an alkyl, an aryl, an alkoxy, an aryloxy, an amino, a hydroxylamino, an alkoxylamino, or a halogen; and
R₉ represents a hydrogen, an alkyl, an aryl, a hydroxyl, an alkoxy, an aryloxy, or an amino.

In general, the present invention provides fluorinated compounds having the general formula (I), (II), or (III):

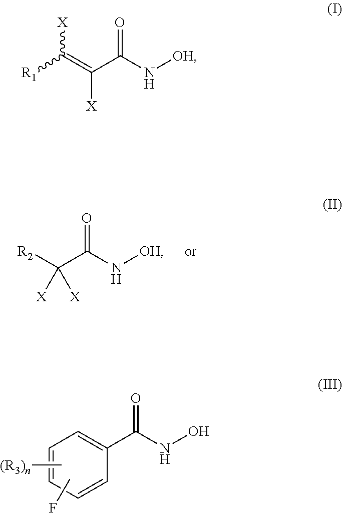

wherein
R₁, R₂, and R₃ are independently a cyclic or acyclic, substituted or unsubstituted aliphatic; a cyclic or acyclic, substituted or unsubstituted heteroaliphatic; a substituted or unsubstituted aryl; or a substituted or unsubstituted heteroaryl;
X is independently H, C₁-C₆ alkyl, or F; with the proviso that at least one X is F;
n is an integer between 1-4, inclusive; and pharmaceutically acceptable salts thereof.

In certain embodiments, the fluorinated compounds are of the general formula (I):

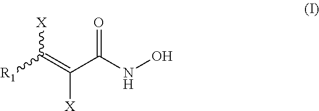

wherein
R₁ is cyclic or acyclic, substituted or unsubstituted aliphatic; cyclic or acyclic, substituted or unsubstituted heteroaliphatic; substituted or unsubstituted aryl, or substituted or unsubstituted heteroaryl;
X is independently H, C₁-C₆ alkyl, or F; with the proviso that at least one X is F; and pharmaceutically acceptable salts thereof.

In certain embodiments, the compound is of the formula (Ia):

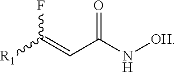

In other embodiments, the compound is of the formula (Ib):

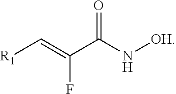

In further embodiments, the compound is of the formula (Ic):

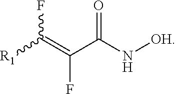

In still further embodiments, the compound is of the formula (Id):

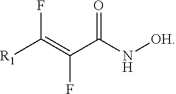

In certain embodiments, the compound is of the formula (Ie):

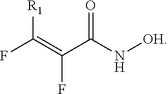

In certain embodiments R₁ is acyclic unsubstituted aliphatic. In other embodiments, R₁ is acyclic substituted aliphatic. In further embodiments, R₁ is cyclic unsubstituted aliphatic. In still further embodiments, R₁ is cyclic unsubstituted aliphatic. In certain embodiments, R₁ is branched substituted or unsubstituted aliphatic. In other embodiments, R₁ is unbranched substituted or unsubstituted aliphatic.

In certain embodiments, R₁ is a substituted or unsubstituted, branched or unbranched alkyl. In other embodiments, R₁ is a substituted or unsubstituted, branched or unbranched C₁₋₁₀ alkyl. In further embodiments, R₁ is substituted or unsubstituted, branched or unbranched C₁₋₆ alkyl. In still further embodiments, R₁ is substituted or unsubstituted, branched or unbranched C₁₋₄ alkyl. In certain embodiments, R₁ is methyl. In other embodiments, R₁ is ethyl. In further embodiments, R₁ is propyl. In still further embodiments, R₁ is butyl.

In certain embodiments, R₁ is a substituted or unsubstituted alkenyl. In other embodiments, R₁ is a substituted or unsubstituted C₂₋₁₀ alkenyl. In further embodiments, R₁ is substituted or unsubstituted C₂₋₆ alkenyl. In still further embodiments, R₁ is substituted or unsubstituted C₂₋₄ alkenyl.

In certain embodiments, R₁ is a substituted or unsubstituted alkynyl. In other embodiments, R₁ is a substituted or unsubstituted C₂₋₁₀ alkynyl. In further embodiments, R₁ is substituted or unsubstituted C₂₋₆ alkynyl. In still further embodiments, R₁ is substituted or unsubstituted C₂₋₄ alkynyl.

In certain embodiments, R₁ contains at least one stereocenter. In other embodiments, R₁ contains 1-5 stereocenters. In further embodiments, R₁ contains 1 stereocenter. In still other embodiments, R₁ contains 2 stereocenters. In certain embodiments, R₁ contains 3 stereocenters. In certain embodiments, the stereocenter has a (R)-configuration. In other embodiments, the stereocenter has a (S)-configuration. In certain embodiments, R₁ does not contain a stereocenter.

In certain embodiments, R₁ is substituted with halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In other embodiments, R₁ is substituted with halogen. In further embodiments, R₁ is substituted with F, Cl, Br, or I.

In certain embodiments, R₁ is substituted with —C(═O)R^{A}; wherein R^{A} is halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In other embodiments, R₁ is substituted with —C(═O)R^{A}; wherein R^{A} is substituted or unsubstituted aryl, arylalkyl, arylalkenyl, or arylalkynyl. In certain embodiments, R₁ is selected from the group consisting of:

wherein

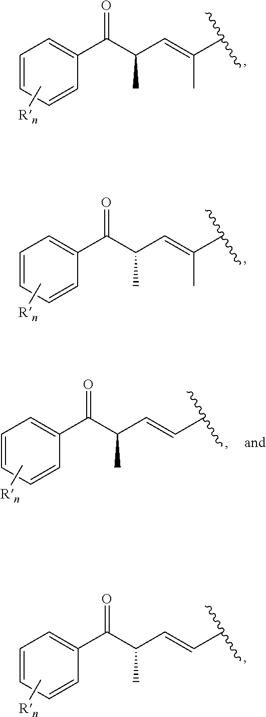

n is an integer between 0-5, inclusive;
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branded or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In other embodiments, R₁ is selected from the group consisting of:

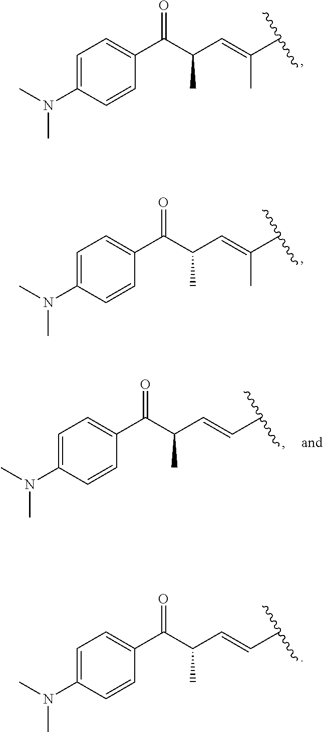 [SMILES: */C(C)=C/[C@@H](C)C(=O)c1ccc(N(C)C)cc1 ; */C(C)=C/[C@H](C)C(=O)c1ccc(N(C)C)cc1 ; */C=C/[C@@H](C)C(=O)c1ccc(N(C)C)cc1 ; */C=C/[C@H](C)C(=O)c1ccc(N(C)C)cc1]

In certain embodiments, R₁ is substituted with —C(═O)R^{A}; wherein R^{A} is substituted or unsubstituted heteroaryl, heteroarylalkyl, heteroarylalkenyl, or heteroarylalkynyl. In other embodiments, R₁ is selected from the group consisting of:

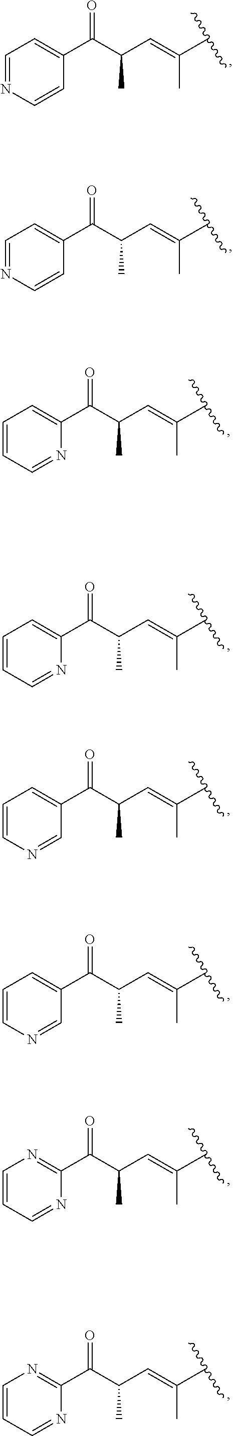

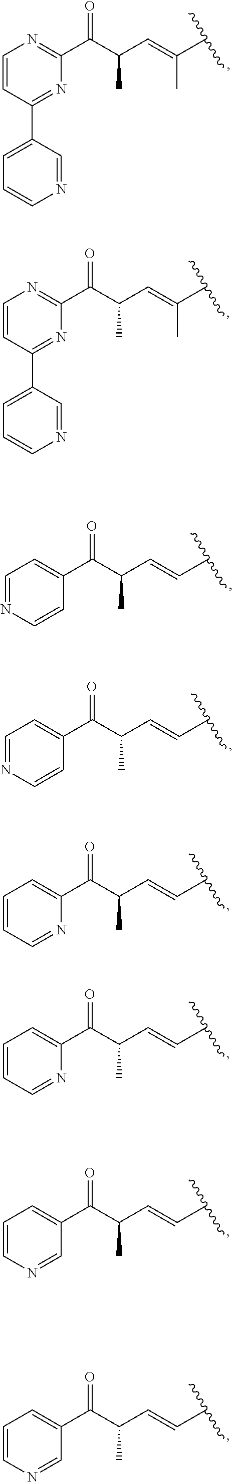

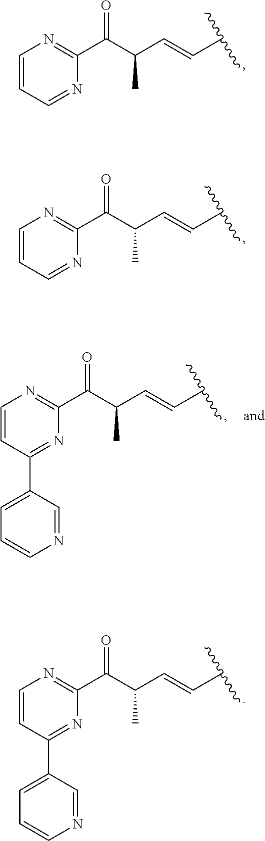 [SMILES: */C=C/[C@@H](C)C(=O)c1ncccn1 ; */C=C/[C@H](C)C(=O)c1ncccn1 ; */C=C/[C@@H](C)C(=O)c1nccc(-c2cccnc2)n1 ; */C=C/[C@H](C)C(=O)c1nccc(-c2cccnc2)n1]

In certain embodiments, R₁ is substituted or unsubstituted aryl. In other embodiments, R₁ is unsubstituted aryl. In further embodiments, R₁ is substituted aryl. In certain embodiments, R₁ is 6-membered aryl. In other embodiments, R₁ is 8-membered aryl. In further embodiments, R₁ is 10-membered aryl. In certain embodiments, R₁ is unsubstituted phenyl. In other embodiments, R₁ is substituted phenyl. In further embodiments, R₁ is monosubstituted phenyl. In certain embodiments, R₁ is disubstituted phenyl. In other embodiments, R₁ is trisubstituted phenyl. In certain embodiments, R₁ is monocyclic ring system. In other embodiments, R₁ is a bicyclic ring system. In further embodiments, R₁ has one aromatic ring. In still further embodiments, R₁ has two aromatic rings. In certain embodiments, R₁ comprises phenyl. In other embodiments, R₁ comprises naphthyl. In further embodiments, R₁ comprises tetrahydronaphthyl.

In certain embodiments, R₁ is

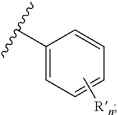

wherein
n is an integer between 0-5, inclusive;
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In certain embodiments, the R′ groups are the same. In other embodiments, the R′ groups are different. In further embodiments, two R′ groups are taken together to form a ring. In certain embodiments, two R′ groups are taken together to form a carbocyclic ring. In other embodiments, two R′ groups are taken together to form a heterocyclic ring. In further embodiments, two R′ groups are taken together to form an aromatic ring. In certain embodiments, two R′ groups are taken together to form an aryl ring. In other embodiments, two R′ groups are taken together to form a heteroaryl ring.

In certain embodiments, n is 1. In other embodiments, n is 2. In further embodiments, n is 3. In still further embodiments, n is 4. In certain embodiments, n is 5.

In certain embodiments, R₁ is selected from the group consisting of:

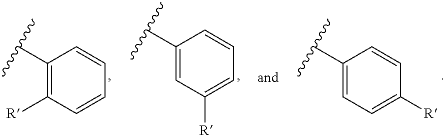

In other embodiments, R′ is selected from the group consisting of:

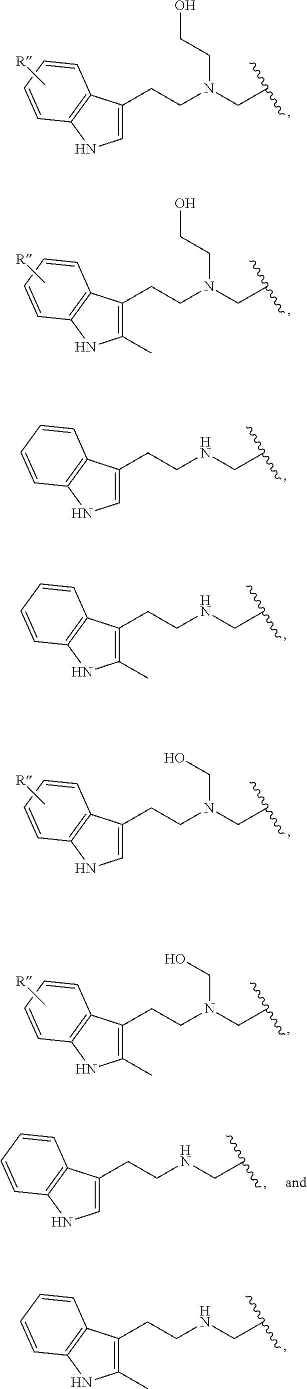

wherein each occurrence of R″ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{C}; —C(═O)R^{C}; —CO₂R^{C}; —C(═O)N(R^{C})₂; —CN; —SCN; —SR^{C}; —SOR^{C}; —SO₂R^{C}; —NO₂; —N(R^{C})₂; —NHC(O)R^{C}; or —C(R^{C})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In further embodiments, R′ is selected from the group consisting of:

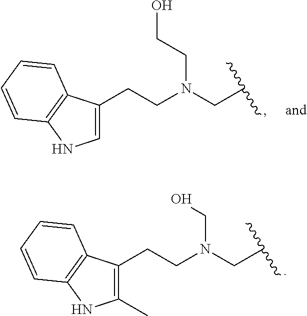 [SMILES: *CN(CCO)CCc1c[nH]c2ccccc12 ; *CN(CO)CCc1c(C)[nH]c2ccccc12]

In other embodiments, R′ is selected from the group consisting of:

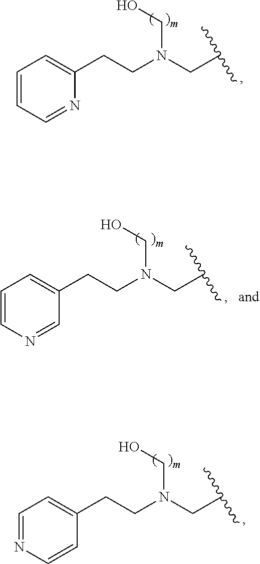

wherein m is an integer between 1 and 15, inclusive.

In further embodiments, R′ is selected from the group consisting of:

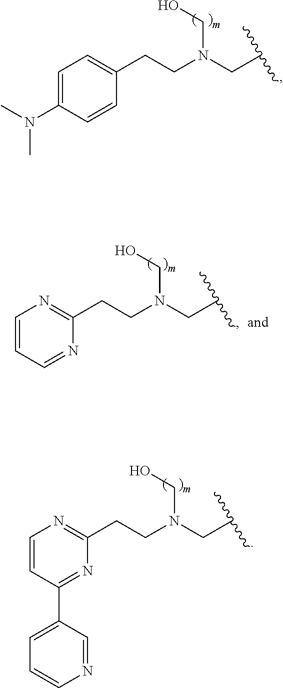

In still further embodiments, R′ is selected from the group consisting of:

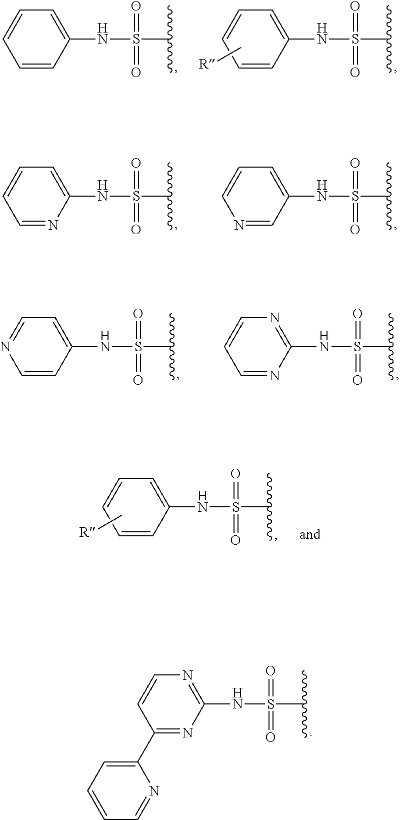

In certain embodiments, R′ is selected from the group consisting of:

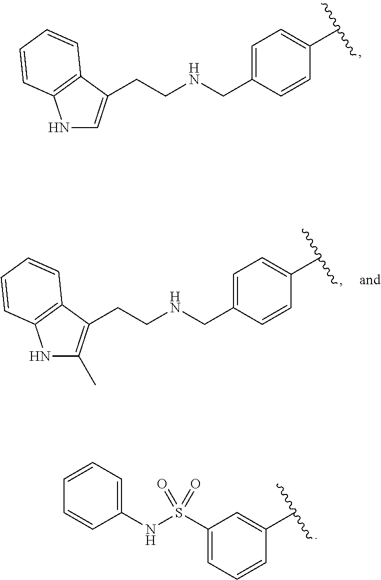 [SMILES: *c1ccc(CNCCc2c[nH]c3ccccc23)cc1 ; *c1ccc(CNCCc2c(C)[nH]c3ccccc23)cc1 ; *c1cccc(S(=O)(=O)Nc2ccccc2)c1]

In certain embodiments, R₁ is selected from the group consisting of:

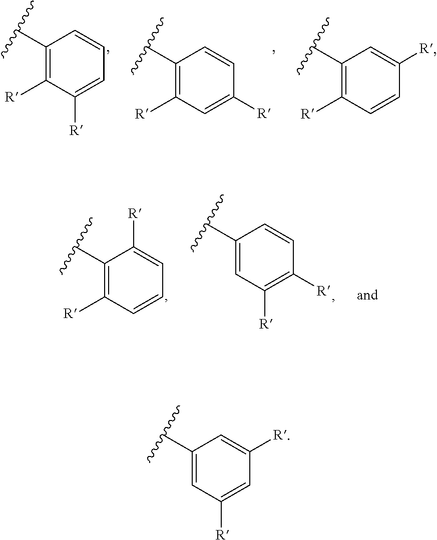

wherein R′ are as defined above.

In other embodiments, R₁ is selected from the group consisting of:

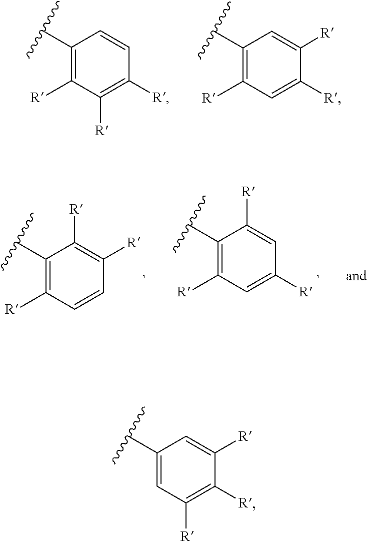

wherein R′ are as defined above.

In certain embodiments, R₁ is substituted or unsubstituted heteroaryl. In other embodiments, R₁ is unsubstituted heteroaryl. In further embodiments, R₁ is substituted heteroaryl. In still further embodiments, R₁ is a nitrogen-containing heretoaryl. In certain embodiments, R₁ is an O-containing heteroaryl. In other embodiments, R₁ is a S-containing heteroaryl. In further embodiments, R₁ is a 5-membered heteroaryl. In certain embodiments, R₁ is a 6-membered heteroaryl. In other embodiments, R₁ is a bicyclic heteroaryl. In further embodiments, R₁ is a tricyclic heteroaryl. In still further embodiments, R₁ is selected from the group consisting of:

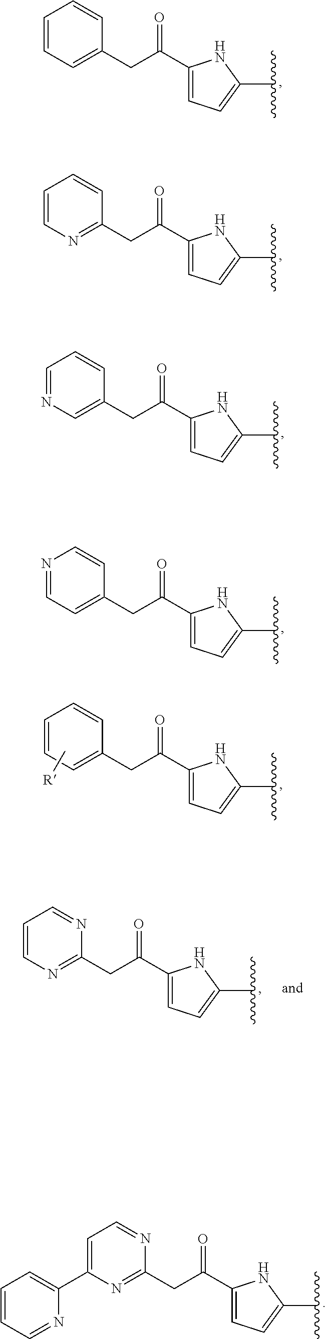

In certain embodiments, R₁ is

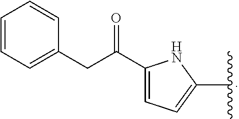 [SMILES: *c1ccc(C(=O)Cc2ccccc2)[nH]1]

In certain embodiments, the fluorinated compounds are of the general formula (II):

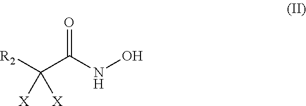

wherein
R₂ is cyclic or acyclic, substituted or unsubstituted aliphatic; cyclic or acyclic, substituted or unsubstituted heteroaliphatic; substituted or unsubstituted aryl, or substituted or unsubstituted heteroaryl;
X is independently H, C₁₋₆ alkyl, or fluorine; with the proviso that at least one X is fluorine; or a pharmaceutically acceptable salt thereof.

In other embodiments, the compound of formula (II) is selected from the group consisting of:

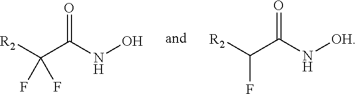

In certain embodiments, R₂ is a cyclic or acyclic, substituted or unsubstituted, branched or unbranched aliphatic. In other embodiments, R₂ is a cyclic, substituted or unsubstituted, branched or unbranched aliphatic. In further embodiments, R₂ is an acyclic, substituted or unsubstituted, branched or unbranched aliphatic. In certain embodiments, R₂ is substituted or unsubstituted, branched or unbranched C₁₋₁₀ alkyl. In other embodiments, R₂ is substituted or unsubstituted, branched or unbranched C₁₋₆ alkyl. In further embodiments, R₂ is substituted or unsubstituted, branched or unbranched C₁₋₄ alkyl. In certain embodiments, R₂ is methyl. In other embodiments, R₂ is ethyl. In further embodiments, R₂ is propyl. In still further embodiments, R₂ is butyl. In certain embodiments, R₂ is substituted or unsubstituted alkenyl. In other embodiments, R₂ is substituted or unsubstituted, C₂₋₁₀ alkenyl. In certain embodiments, R₂ is substituted or unsubstituted, C₂₋₆ alkenyl. In other embodiments, R₂ is substituted or unsubstituted, C₂₋₄ alkenyl. In certain embodiments, R₂ is ethenyl. In other embodiments, R₂ is propenyl. In further embodiment, R₂ is butenyl. In certain embodiments, R₂ is substituted or unsubstituted alkynyl. In other embodiments, R₂ is substituted or unsubstituted, C₂₋₁₀ alkynyl. In certain embodiments, R₂ is substituted or unsubstituted, C₂₋₆ alkynyl. In other embodiments, R₂ is substituted or unsubstituted, C₂₋₄ alkynyl. In certain embodiments, R₂ is ethynyl. In other embodiments, R₂ is propynyl. In further embodiment, R₂ is butynyl.

In certain embodiments, R₂ contains at least one stereocenter. In other embodiments, R₂ contains 1-5 stereocenters. In further embodiments, R₂ contains 1 stereocenter. In still other embodiments, R₂ contains 2 stereocenters. In certain embodiments, R₂ contains 3 stereocenters. In certain embodiments, the stereocenter has a (R)-configuration. In other embodiments, the stereocenter has a (S)-configuration. In certain embodiments, R₂ does not contain a stereocenter.

In certain embodiments, R₂ is a cyclic or acyclic, substituted or unsubstituted, branched or unbranched heteroaliphatic. In other embodiments, R₂ is a cyclic, substituted or unsubstituted, branched or unbranched heteroaliphatic. In further embodiments, R₂ is an acyclic, substituted or unsubstituted, branched or unbranched heteroaliphatic. In certain embodiments, R₂ is substituted C₁₋₁₀ alkyl. In other embodiments, R₂ is substituted C₁₋₆ alkyl. In further embodiments, R₂ is substituted C₁₋₄ alkyl.

In certain embodiments, R₂ is substituted with —C(O)R′″, wherein R′″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In other embodiments, R₂ is substituted with one of the following moieties:

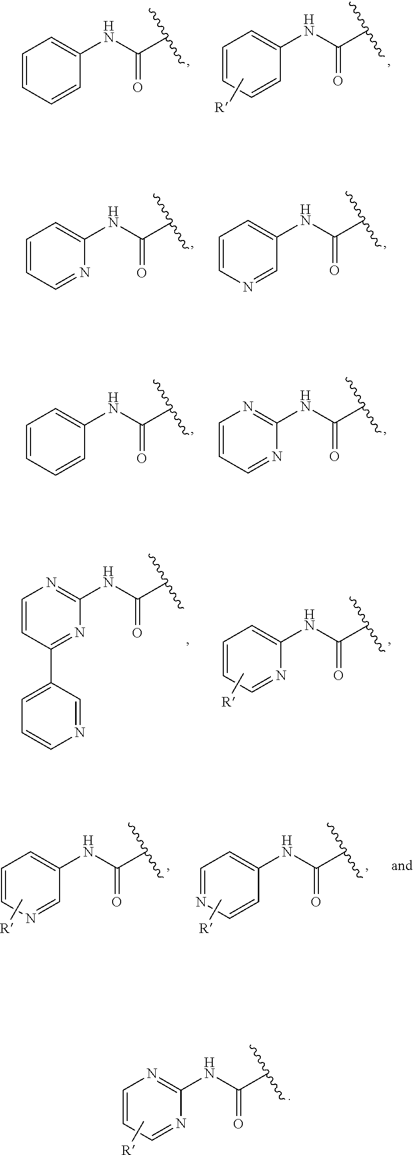

In further embodiments, R₂ is selected from the group consisting of:

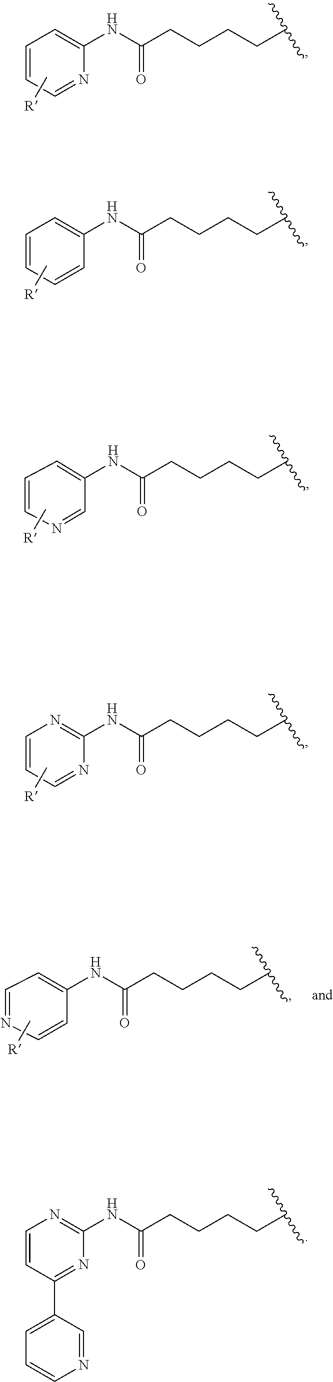

In still further embodiments, R₂ is selected from the group consisting of:

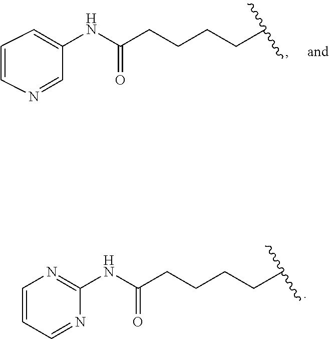 [SMILES: *CCCCC(=O)Nc1cccnc1 ; *CCCCC(=O)Nc1ncccn1]

In certain embodiments, R₂ is:

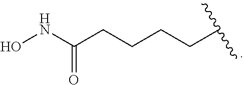 [SMILES: *CCCCC(=O)NO]

In certain embodiments, R₂ is selected from the group consisting of:

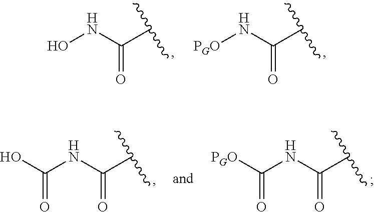

wherein P_{G} is an O protecting group. In certain embodiments, P_{G} is alkyl, aryl arylalkyl, arylalkenyl, arylalkynyl, heteroaryl, heteroarylalkyl, heteroarylalkenyl, or heteroarylalkynyl. In other embodiments, —OP_{G} is selected from the group consisting of substituted alkyl ethers, substituted benzyl ethers, and silyl ethers. In further embodiments, —OP_{G} is an ester. In still further embodiments, —OPG is a carbonate or a sulfonate.

In certain embodiments, R₂ is substituted with a substituted or unsubstituted aryl. In other embodiments, R₂ is substituted with a substituted or unsubstituted heterocycle. In further embodiments, R₂ is substituted with a monocyclic moiety. In still further embodiments, R₂ is substituted with a bicyclic moiety. In other embodiments, R₂ is substituted with a tricyclic moiety.

In certain embodiments, R₂ is substituted with one of the following moieties:

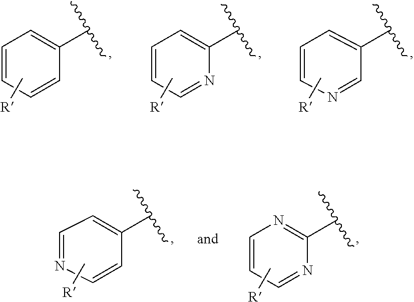

wherein R′ is as described above. In other embodiments, R₂ is

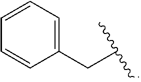 [SMILES: *Cc1ccccc1]

In further embodiments, R₂ is substituted with

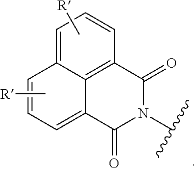

In still further embodiments, R₂ is

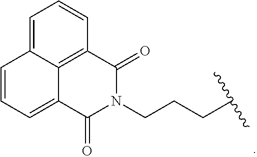 [SMILES: *CCCN1C(=O)c2cccc3cccc(c23)C1=O]

In certain embodiments, the fluorinated compounds are N-hydroxy-fluoro-benzamides of the general formula (III):

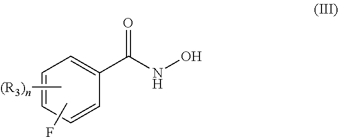

wherein
R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy;
X is independently H, C₁-C₆ alkyl, or fluorine; with the proviso that at least one X is fluorine;
n is an integer between 1-4, inclusive; or a pharmaceutically acceptable salt thereof.

In other embodiments, the compound of the formula (III) is selected from the group consisting of:

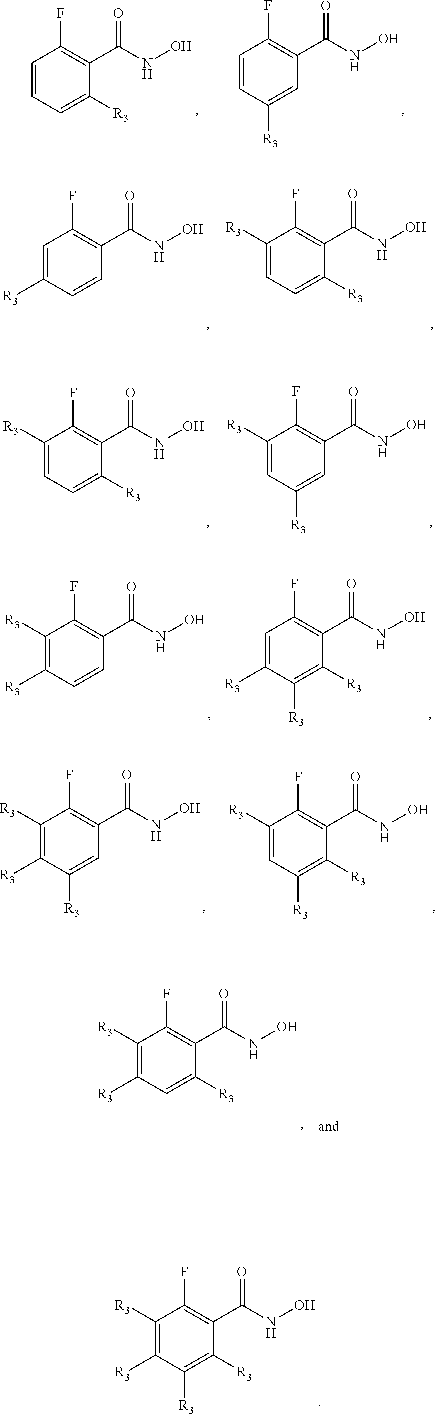

In further embodiments, the compound of the formula (III) is selected from the group consisting of:

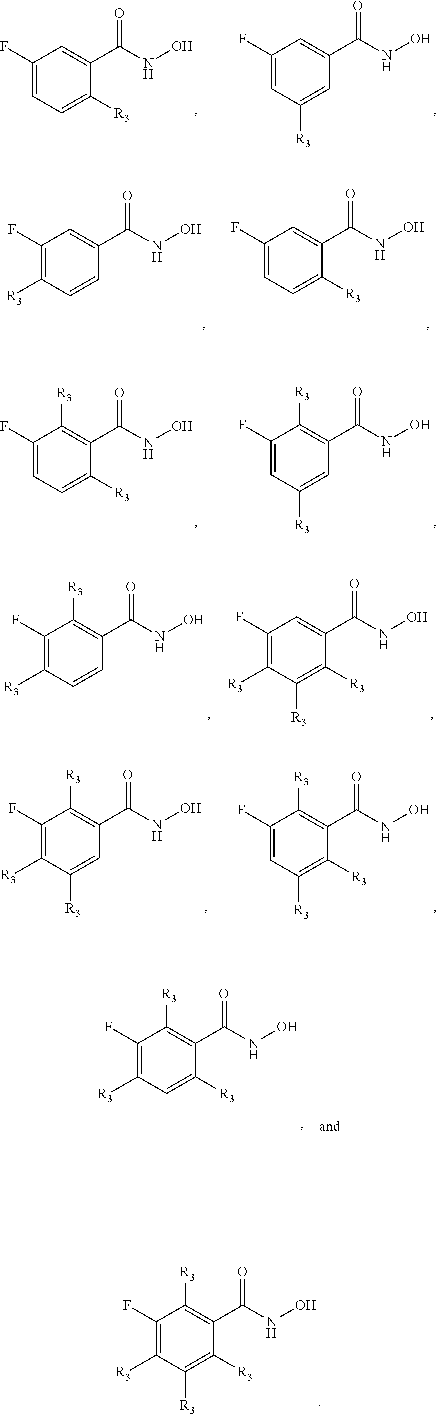

In still further embodiments, the compound of the formula (III) is selected from the group consisting of:

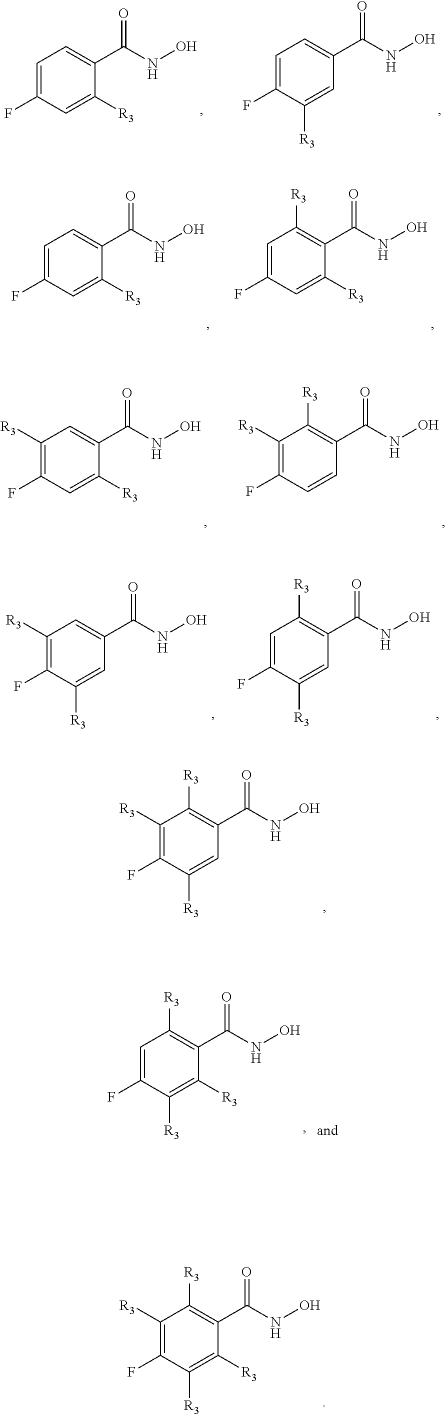

In certain embodiments, the compound of the formula (III) is selected from the group consisting of:

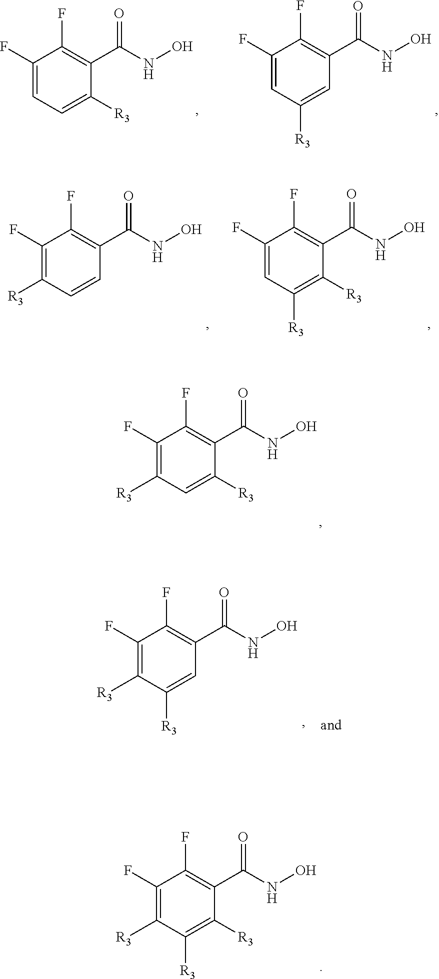

In other embodiments, the compound of the formula (III) is selected from the group consisting of:

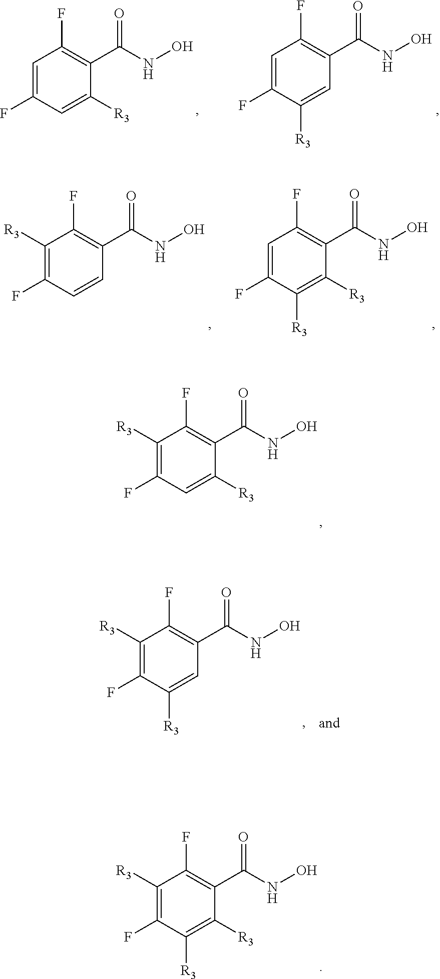

In further embodiments, the compound of the formula (III) is selected from the group consisting of:

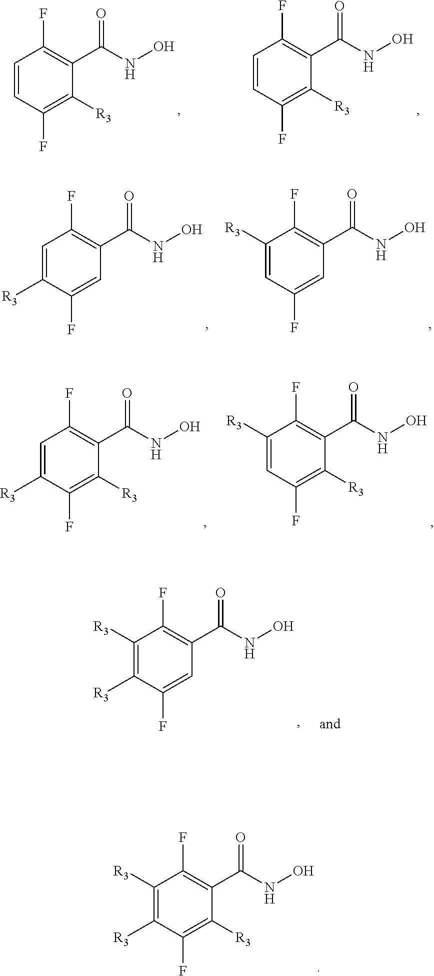

In certain embodiments, the compound of the formula (III) is selected from the group consisting of:

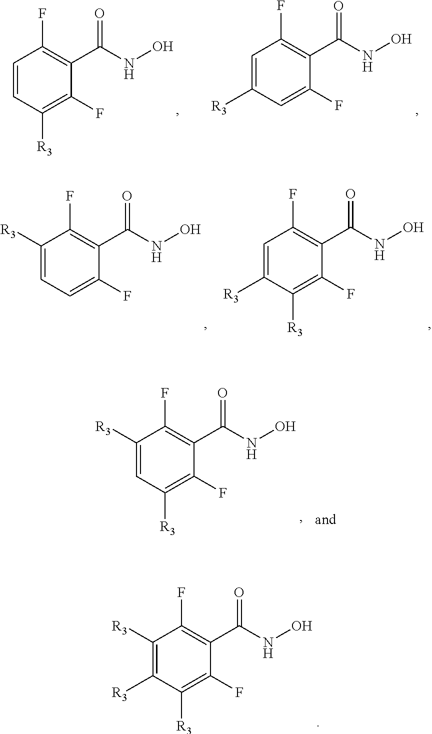

In other embodiments, the compound of the formula (III) is selected from the group consisting of:

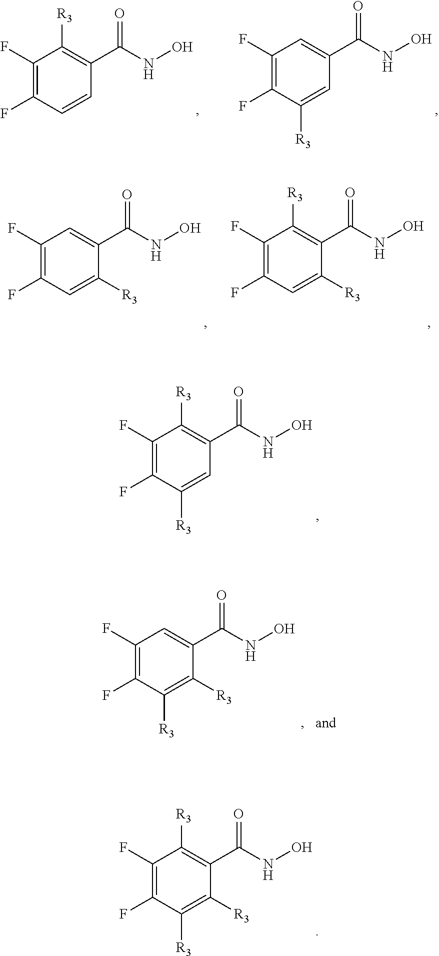

In further embodiments, the compound of the formula (III) is selected from the group consisting of:

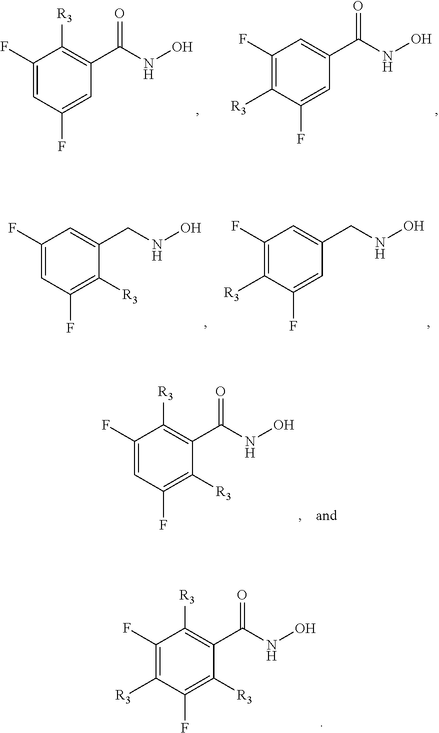

In still further embodiments, the compound of the formula (III) is selected from the group consisting of:

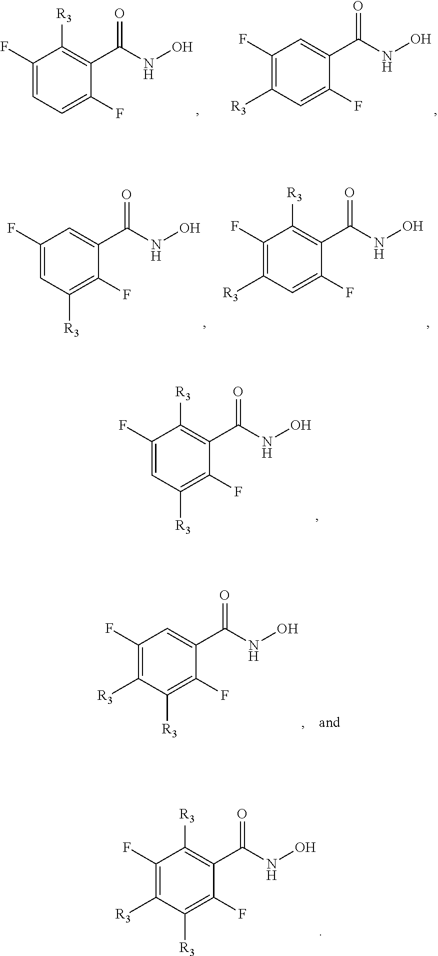

In certain embodiments, the compound of the formula (III) is selected from the group consisting of:

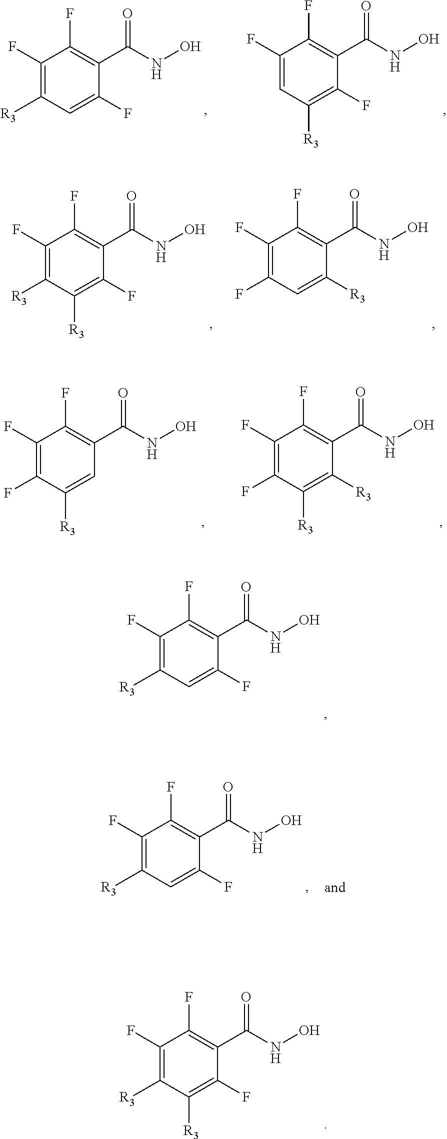

In other embodiments, the compound of the formula (III) is selected from the group consisting of:

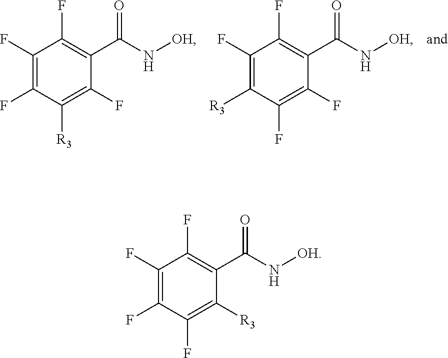

In certain embodiments, wherein R₃ is cyclic or acyclic, substituted or unsubstituted, branched or unbranched aliphatic. In other embodiments, R₃ is cyclic, substituted or unsubstituted, branched or unbranched aliphatic. In further embodiments, R₃ is an acyclic, substituted or unsubstituted, branched or unbranched aliphatic. In certain embodiments, R₃ is substituted or unsubstituted alkyl. In other embodiments, R₃ is substituted or unsubstituted C₁₋₁₀ alkyl. In further embodiments, R₃ is substituted or unsubstituted C₁₋₆ alkyl. In still further embodiments, R₃ is substituted or unsubstituted C₁₋₄ alkyl. In certain embodiments, R₃ is substituted or unsubstituted alkenyl. In other embodiments, R₃ is substituted or unsubstituted C₂₋₁₀ alkenyl. In further embodiments, R₃ is substituted or unsubstituted C₂₋₆ alkenyl. In further embodiments, R₃ is substituted or unsubstituted C₂₋₄ alkenyl.

In certain embodiments, R₃ contains at least one stereocenter. In other embodiments, R₃ contains 1-5 stereocenters. In further embodiments, R₃ contains 1 stereocenter. In still other embodiments, R₃ contains 2 stereocenters. In certain embodiments, R₃ contains 3 stereocenters. In certain embodiments, the stereocenter has a (R)-configuration. In other embodiments, the stereocenter has a (S)-configuration. In certain embodiments, R₃ does not contain a stereocenter.

In certain embodiments, R₃ is a cyclic or acyclic, substituted or unsubstituted, branched or unbranched heteroaliphatic. In other embodiments, wherein R₃ is cyclic, substituted or unsubstituted, branched or unbranched heteroaliphatic. In further embodiments, R₃ is acyclic, substituted or unsubstituted, branched or unbranched heteroaliphatic.

In certain embodiments, R₃ is substituted with a substituted or unsubstituted aryl. In other embodiments, R₃ is substituted with a substituted or unsubstituted heterocyclic. In further embodiments, R₃ is substituted with a monocyclic moiety. In still further embodiments, R₃ is substituted with a bicyclic moiety. In other embodiments, R₃ is substituted with a tricyclic moiety.

In certain embodiments, R₃ is substituted with

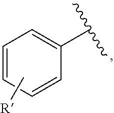

wherein R′ is as described above. In certain embodiments, R′ is at the para-position.

In certain embodiments, R₃ is a substituted or unsubstituted heteroaryl. In other embodiments, R₃ is a unsubstituted heteroaryl. In further embodiments, R₃ is a substituted heteroaryl. In still further embodiments, R₃ is N-containing heteroaryl. In certain embodiments, R₃ is O-containing heteroaryl. In other embodiments, R₃ is S-containing heteroaryl. In further embodiments, R₃ is 5-membered heteroaryl. In certain embodiments, R₃ is 6-membered heteroaryl. In other embodiments, R₃ is bicyclic heteroaryl. In further embodiments, R₃ is tricyclic heteroaryl. In certain embodiments, R₃ is substituted with one of the following moieties:

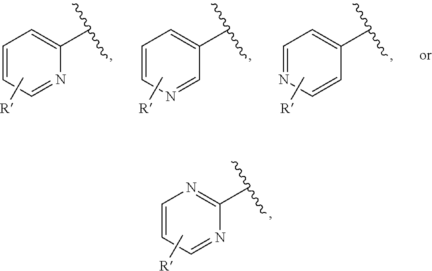

wherein R′ is as described above.

In certain embodiments, R₃ is substituted with —C(O)R″, wherein R″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In other embodiments, R₃ is substituted with —NHC(O)₂R″, wherein R″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In certain embodiments, R₃ is substituted with:

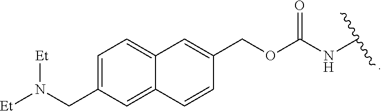 [SMILES: *NC(=O)OCc1ccc2cc(CN(CC)CC)ccc2c1]

In other embodiments, R₃ is substituted with —C(O)R″, wherein R″ is —N(R^{B})₂; or —NHC(O)R^{B}; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In other embodiments, R₃ is substituted with

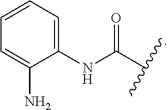 [SMILES: *C(=O)Nc1ccccc1N]

In other embodiments, R₃ is selected from the group consisting of:

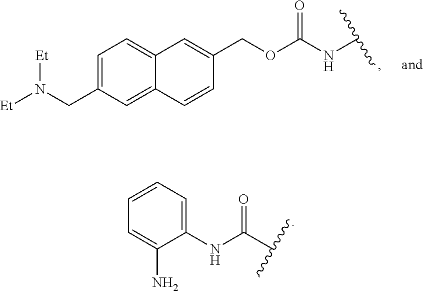 [SMILES: *NC(=O)OCc1ccc2cc(CN(CC)CC)ccc2c1 ; *C(=O)Nc1ccccc1N]

In certain embodiments, the compound is

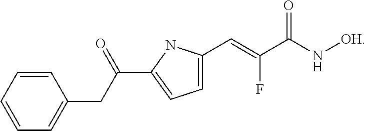 [SMILES: O=C(NO)/C(F)=C/c1ccc(C(=O)Cc2ccccc2)[nH]1]

In certain embodiments, the compound

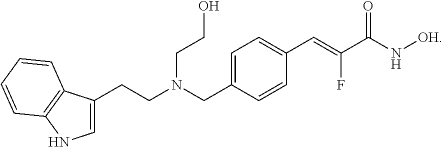 [SMILES: O=C(NO)/C(F)=C/c1ccc(CN(CCO)CCc2c[nH]c3ccccc23)cc1]

In certain embodiments, the compound is

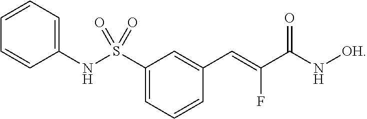 [SMILES: O=C(NO)/C(F)=C/c1cccc(S(=O)(=O)Nc2ccccc2)c1]

In certain embodiments, the compound

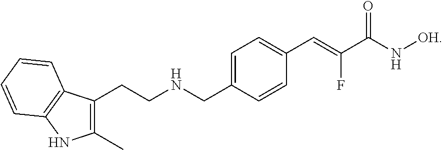 [SMILES: Cc1[nH]c2ccccc2c1CCNCc1ccc(/C=C(\F)C(=O)NO)cc1]

In certain embodiments, the compound is

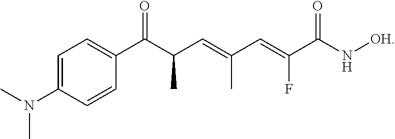 [SMILES: CC(/C=C(\F)C(=O)NO)=C\[C@@H](C)C(=O)c1ccc(N(C)C)cc1]

In certain embodiments, the compound is

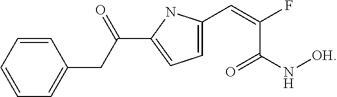 [SMILES: O=C(NO)/C(F)=C\c1ccc(C(=O)Cc2ccccc2)[nH]1]

In certain embodiments, the compound is

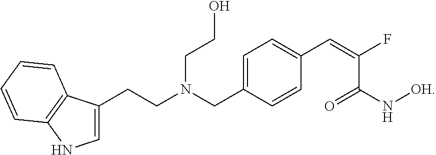 [SMILES: O=C(NO)/C(F)=C\c1ccc(CN(CCO)CCc2c[nH]c3ccccc23)cc1]

In certain embodiments, the compound is

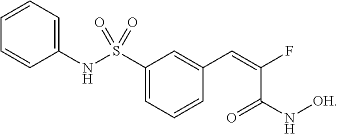 [SMILES: O=C(NO)/C(F)=C\c1cccc(S(=O)(=O)Nc2ccccc2)c1]

In certain embodiments, the compound is

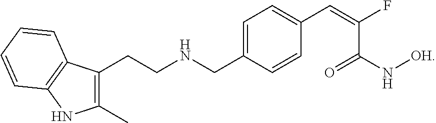 [SMILES: Cc1[nH]c2ccccc2c1CCNCc1ccc(/C=C(/F)C(=O)NO)cc1]

In certain embodiments, the compound is

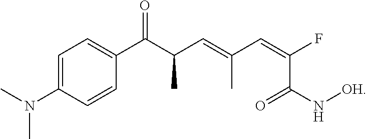 [SMILES: CC(=C\[C@@H](C)C(=O)c1ccc(N(C)C)cc1)/C=C(/F)C(=O)NO]

In other embodiments, the compound is

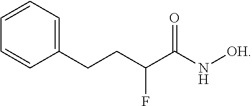 [SMILES: O=C(NO)C(F)CCc1ccccc1]

In certain embodiments, the compound is

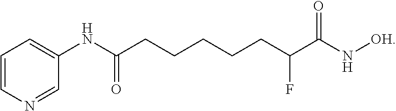 [SMILES: O=C(CCCCCC(F)C(=O)NO)Nc1cccnc1]

In certain embodiments, the compound is

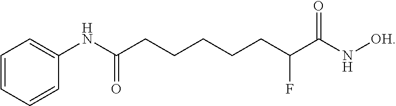 [SMILES: O=C(CCCCCC(F)C(=O)NO)Nc1ccccc1]

In certain embodiments, the compound is

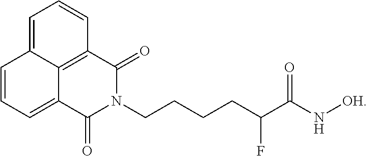 [SMILES: O=C(NO)C(F)CCCCN1C(=O)c2cccc3cccc(c23)C1=O]

In certain embodiments, the compound is

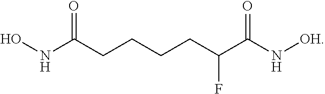 [SMILES: O=C(CCCCC(F)C(=O)NO)NO]

In certain embodiments, the compound is

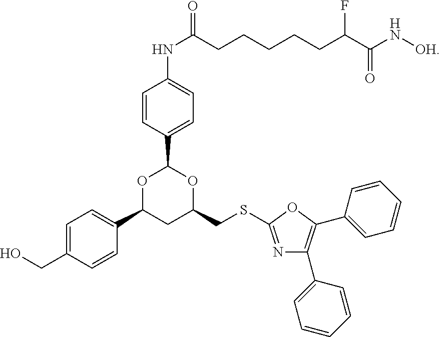 [SMILES: O=C(CCCCCC(F)C(=O)NO)Nc1ccc([C@H]2O[C@@H](CSc3nc(-c4ccccc4)c(-c4ccccc4)o3)C[C@@H](c3ccc(CO)cc3)O2)cc1]

In certain embodiments, the compound is

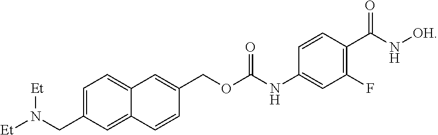 [SMILES: CCN(CC)Cc1ccc2cc(COC(=O)Nc3ccc(C(=O)NO)c(F)c3)ccc2c1]

In certain embodiments, the compound is

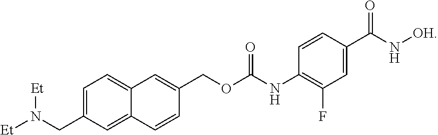 [SMILES: CCN(CC)Cc1ccc2cc(COC(=O)Nc3ccc(C(=O)NO)cc3F)ccc2c1]

In certain embodiments, the compound is O

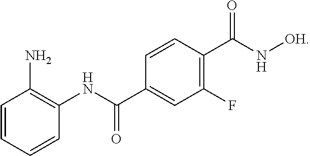 [SMILES: Nc1ccccc1NC(=O)c1ccc(C(=O)NO)c(F)c1]

In certain embodiments, the compound is

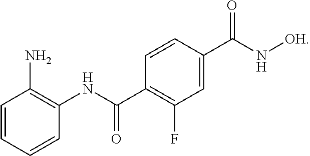 [SMILES: Nc1ccccc1NC(=O)c1ccc(C(=O)NO)cc1F]

### Pharmaceutical Compositions

The present invention provides novel compounds useful in the treatment of diseases or disorders associated with HDAC activity. The compounds are useful in the treatment of diseases or condition that benefit from inhibition of deacetylation activity (e.g., HDAC inhibition, TDAC inhibition). In certain embodiments, the inventive compounds are useful in the treatment of proliferative diseases, such as cancer (e.g., cutaneous T-cell lymphoma, peripheral T-cell lymphoma) or benign proliferative diseases; autoimmune diseases; allergic and inflammatory diseases; diseases of the central nervous system (CNS), such as neurodegenerative diseases (e.g. Huntington's disease); vascular diseases, such as restenosis; musculoskeletal diseases; cardiovascular diseases, such as stroke; pulmonary diseases; gastric diseases; genetic diseases; and infectious diseases. Class- or isoform-specific HDAC inhibitors may be particularly useful in the treatment of disease or disorders associated with aberrant HDAC activity from a particular Class or isoform. For example, Class IIa HDAC inhibitors may be useful in the treatment of autoimmune or allergic diseases, cardiovascular diseases, or neurodegenerative diseases since Class IIa HDACs have been suggested to play a role in immune tolerance, cardiac remodeling, and neuronal death.

Accordingly, in another aspect of the present invention, pharmaceutical compositions are provided, which comprise any one of the compounds described herein (or a prodrug, pharmaceutically acceptable salt or other pharmaceutically acceptable derivative thereof) and optionally a pharmaceutically acceptable excipient. In certain embodiments, these compositions optionally further comprise one or more additional therapeutic agents. Alternatively, a compound of this invention may be administered to a patient in need thereof in combination with the administration of one or more other therapeutic agents. For example, in the treatment of cancer, an additional therapeutic agents for conjoint administration or inclusion in a pharmaceutical composition with a compound of this invention may be an approved chemotherapeutic agent.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As described above, the pharmaceutical compositions of the present invention optionally comprise a pharmaceutically acceptable excipient, which, as used herein, includes any and all solvents, diluents, or other liquid vehicle, dispersion or suspension aids, surface active agents, isotonic agents, thickening or emulsifying agents, preservatives, antioxidants, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar, buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monosteamte, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols, and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation,” as used herein, means any formulation which is pharmaceutically acceptable for intradermal administration of a compound of the invention by application of the formulation to the epidermis. In certain embodiments of the invention, the topical formulation comprises a excipient system. Pharmaceutically effective excipients include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other excipient known in the art for topically administering pharmaceuticals. A more complete listing of art-known carvers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyarrisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum coreum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone. In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable excipient and any needed preservatives or buffers as may be required. Ophthalmic formulations, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix (e.g., PLGA) or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent or anticancer agent), or they may achieve different effects (e.g., control of any adverse effects).

For example, other therapies or anticancer agents that may be used in combination with the inventive compounds of the present invention for cancer therapy include surgery, radiotherapy (in but a few examples, α-radiation, neutron beam radiotherapy, electron beam radiotherapy, proton therapy, brachytherapy, and systemic radioactive isotopes, to name a few), endocrine therapy, biologic response modifiers (interferon, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ion (Cisplatin, Carboplatin), enzymes (Asparaginase), and hormones (Tamoxifen, Leuprelide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/draglis&ame).

In certain embodiments, the pharmaceutical compositions of the present invention further comprise one or more additional therapeutically active ingredients (e.g., chemotherapeutic and/or palliative). For purposes of the invention, the term “palliative” refer, to treatment that is focused on the relief of symptoms of a disease and/or side effects of a therapeutic regimen, but is not curative. For example, palliative treatment encompasses painkillers, antinausea medication and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

Additionally, the present invention provides pharmaceutically acceptable derivatives of the inventive compounds, and methods of treating a subject using these compounds, pharmaceutical compositions thereof, or either of these in combination with one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

### Treatment Kit

In certain embodiments, the present invention relates to a kit for conveniently and effectively carrying out the methods in accordance with the present invention. In general, the pharmaceutical pack or kit comprises one or more containers filled with one or more of the ingredients of the inventive compounds or pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Pharmaceutical Uses and Methods of Treatment

In general, methods of using the compounds of the present invention comprise administering to a subject in need thereof a therapeutically effective amount of a compound of the present invention. The compounds of the invention are generally inhibitors of deacetylase activity. As discussed above, the compounds of the invention are typically inhibitors of histone deacetylases and, as such, are useful in the treatment of disorders modulated by histone deacetylases. Diseases associated with a particular HDAC class or isoform may be treated by an inventive compound that specifically inhibits that particular class or isoform. Other deacetylases such as tubulin deacetylases may also be inhibited by the inventive compounds.

In certain embodiments, compounds of the invention are useful in the treatment of proliferative diseases (e.g., cancer, benign neoplasms, inflammatory disease, autoimmune diseases). In other embodiments, the inventive compounds are useful in the treatment of autoimmune diseases; allergic and inflammatory diseases; diseases of the central nervous system (CNS), such as neurodegenerative diseases (e.g. Huntington's disease); vascular diseases, such as restenosis; musculoskeletal diseases; cardiovascular diseases, such as stroke; pulmonary diseases; gastric diseases; genetic diseases; and infectious diseases.

In another aspect of the invention, methods for the treatment of cancer are provided comprising administering a therapeutically effective amount of an inventive compound, as described herein, to a subject in need thereof. In certain embodiments, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments, the inventive compound is administered parenterally. In certain embodiments, the inventive compound is administered intravenously. In certain embodiments, the inventive compound is administered topically. In certain embodiments of the present invention, a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells,” as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like.

In certain embodiments, the method involves the administration of a therapeutically effective amount of the compound or a pharmaceutically acceptable derivative thereof to a subject (including, but not limited to a human or animal) in need of it. In certain embodiments, the inventive compounds as useful for the treatment of cancer (including, but not limited to, glioblastoma, retinoblastoma, breast cancer, cervical cancer, colon and rectal cancer, leukemia, lymphoma, lung cancer (including, but not limited to, small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer).

In certain embodiments, the inventive anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors.

In certain embodiments, the inventive compounds also find use in the prevention of restenosis of blood vessels subject to traumas such as angioplasty and stenting. For example, it is contemplated that the compounds of the invention may be useful as a coating for implanted medical devices, such as tubings, shunts, catheters, artificial implants, pins, electrical implants such as pacemakers, and especially for arterial or venous stents, including balloon-expandable stents. In certain embodiments inventive compounds may be bound to an implantable medical device, or alternatively, may be passively adsorbed to the surface of the implantable device. In certain other embodiments, the inventive compounds may be formulated to be contained within, or, adapted to release by a surgical or medical device or implant, such as, for example, stents, sutures, indwelling catheters, prosthesis, and the like. For example, drugs having antiproliferative and/or anti-inflammatory activities have been evaluated as stent coatings, and have shown promise in preventing retenosis (See, for example, Presbitero et al., “Drug eluting stents do they make the difference?”, Minerva Cardioangiol., 2002, 50(5):431-442; Ruygrok et al., “Rapamycin in cardiovascular medicine”, Intern. Med. J., 2003, 33(3):103-109; and Marx et al., “Bench to bedside: the development of rapamycin and its application to stent restenosis”, Circulation, 2001, 104(8):852-855, each of these references is incorporated herein by reference in its entirety). Accordingly, without wishing to be bound to any particular theory, Applicant proposes that inventive compounds having antiproliferative effects can be used as stent coatings and/or in stent drug delivery devices, inter alia for the prevention of restenosis or reduction of restenosis rate. Suitable coatings and the general preparation of coated implantable devices are described in U.S. Pat. Nos. 6,099,562; 5,886,026; and 5,304,121; each of which is incorporated herein by reference. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. A variety of compositions and methods related to stem coating and/or local stent drug delivery for preventing restenosis are known in the art (see, for example, U.S. Pat. Nos. 6,517,889; 6,273,913; 6,258,121; 6,251,136; 6,248,127; 6,231,600; 6,203,551; 6,153,252; 6,071,305; 5,891,507; 5,837,313 and U.S. Patent Application Publication No.: 2001/10027340, each of which is incorporated herein by reference in its entirety). For example, stents may be coated with polymer-drug conjugates by dipping the stent in polymer-drug solution or spraying the stent with such a solution. In certain embodiment, suitable materials for the implantable device include biocompatible and nontoxic materials, and maybe chosen from the metals such as nickel-titanium alloys, steel, or biocompatible polymers, hydrogels, polyurethanes, polyethylenes, ethylenevinyl acetate copolymers, etc. In certain embodiments, the inventive compound is coated onto a stent for insertion into an artery or vein following balloon angioplasty.

The compounds of this invention or pharmaceutically acceptable compositions thereof may also be incorporated into compositions for coating implantable medical devices, such as prostheses, artificial valves, vascular grafts, stents, and catheters. Accordingly, the present invention, in another aspect, includes a composition for coating an implantable device comprising a compound of the present invention as described generally above, and in classes and subclasses herein, and a excipient suitable for coating said implantable device. In still another aspect, the present invention includes an implantable device coated with a composition comprising a compound of the present invention as described generally above, and in classes and subclasses herein, and a excipient suitable for coating said implantable device.

Within other aspects of the present invention, methods are provided for expanding the lumen of a body passageway, comprising inserting a stent into the passageway, the stent having a generally tubular structure, the surface of the structure being coated with (or otherwise adapted to release) an inventive compound or composition, such that the passageway is expanded. In certain embodiments, the lumen of a body passageway is expanded in order to eliminate a biliary, gastrointestinal, esophageal, tracheal/bronchial, urethral, and/or vascular obstruction.

Methods for eliminating biliary, gastrointestinal, esophageal, tracheal/bronchial, urethral and/or vascular obstructions using stents are known in the art. The skilled practitioner will know how to adapt these methods in practicing the present invention. For example, guidance can be found in US. Patent Application Publication No.: 2003/0004209 in paragraphs [0146]-[0155], which paragraphs are incorporated herein by reference.

Another aspect of the invention relates to a method for inhibiting the growth of multidrug resistant cells in a biological sample or a patient, which method comprises administering to the patient, or contacting said biological sample with a compound of formula I, II, or III, or a composition comprising said compound.

Additionally, the present invention provides pharmaceutically acceptable derivatives of the inventive compounds, and methods of treating a subject using such compounds, pharmaceutical compositions thereof, or either of these in combination with one or more additional therapeutic agents.

Another aspect of the invention relates to a method of treating or lessening the severity of a disease or condition associated with a proliferative disorder in a patient, said method comprising a step of administering to said patient, a compound of formula A-B—C, I, II, or III, or a composition comprising said compound.

The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, mute of administration, and rate of excretion of the specific compound employed; the duration of the treatment; drugs used in combination or coincidental with the specific compound employed; and like factors well known in the medical arts (see, for example, Goodman and Gilman's The Pharmacological Basis of Therapeutics, Tenth Edition, A. Gilman, J. Hardman and L. Limbird, eds., McGraw-Bill Press, 155-173, 2001, which is incorporated herein by reference in its entirety).

Another aspect of the invention relates to a method for inhibiting histone deacetylase activity in a biological sample or a patient, which method comprises administering to the patient, or contacting said biological sample with an inventive compound or a composition comprising said compound.

Furthermore, after formulation with an appropriate pharmaceutically acceptable excipient in a desired dosage, the pharmaceutical compositions of this invention can be administered to humans and other animals orally, rectally, parenterally, intracisternally, intravaginally, intraperitoneally, topically (as by powders, ointments, creams or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

### Other Uses

The present invention provides novel compounds useful in the treatment of diseases or disorders associated with HDAC activity. The compounds are useful in the treatment of diseases or condition that benefit from inhibition of deacetylation activity (e.g., HDAC inhibition). In particular, the compounds are useful in treating diseases that benefit from inhibiting a particular HDAC isoform or class of HDACs. In certain embodiments, the inventive compounds are useful in the treatment of cellular proliferative diseases, such as cancer (e.g., cutaneous T-cell lymphoma) or benign proliferative diseases; autoimmune diseases; allergic and inflammatory diseases; diseases of the central nervous system (CNS), such as neurodegenerative diseases (e.g. Huntington's disease); vascular diseases, such as restenosis; musculoskeletal diseases; cardiovascular diseases; stroke; pulmonary diseases; gastric diseases; and infectious diseases.

In certain embodiments, the compounds of the present invention are useful as inhibitors of histone deacetylases and thus are useful as antiproliferative agents, and thus may be useful in the treatment of cancer, by effecting tumor cell death or inhibiting the growth of tumor cells. In certain exemplary embodiments, the inventive compounds are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and myeloma cells, and thus are useful for the treatment of leukemias (e.g., myeloid, lymphocytic, myelocytic and lymphoblastic leukemias) and malignant melanomas. In certain embodiments, the inventive compounds are active against cutaneous T-cell lymphoma. Additionally, as described hereein, the inventive compounds may also be useful in the treatment of protozoal infections. Additionally, as described herein, the inventive compounds may also be useful in the treatment of autoimmune or inflammatory diseases. Furthermore, as described herein, the inventive compounds may also be useful in the treatment of neurodegenerative diseases. As described herein, the inventive compounds may also be useful in the treatment of cardiovascular diseases. In certain exemplary embodiments, the compounds of the invention are useful for disorders resulting from protein deacetylation activity or reduced protein acetylation. In certain exemplary embodiments, the compounds of the invention are useful for disorders resulting from histone deacetylation activity or reduced histone acetylation.

Uses according to the present invention, the inventive compounds may be assayed in any of the available assays known in the art for identifying compounds having antiprotozoal, HDAC inhibitory, hair growth, androgen signaling inhibitory, estrogen signaling inhibitory, antiinflammatory activity, and/or antiproliferative activity. For example, the assay may be cellular or non-cellular, in vivo or in vitro, high- or low-throughput format, etc.

Thus, in one aspect, compounds of this invention which are of particular interest include those which:
exhibit HDAC inhibitory activity;
exhibit HDAC Class I inhibitory activity (e.g., HDAC1, HDAC2, HDAC3, HDAC8);
exhibit HDAC Class II inhibitory activity (e.g., HDAC4, HDAC5, HDAC6, HDAC7, HDAC9a, HDAC9b, HDRP/HDAC9c, HDAC10);
exhibit HDAC Class IIa inhibitory activity (e.g., HDAC4, HDAC5, HDAC7, HDAC9a, HDAC9b, HDRP/HDAC9c);
exhibit HDAC Class IIb inhibitory activity (e.g., HDAC6, HDAC10);
exhibit HDAC Class III inhibitory activity (e.g., SIRT1-7);
exhibit HDAC Class IV inhibitory activity (e.g., HDAC 11);
exhibit sirtuin inhibitory activity (e.g., SIRT1, SIRT2, SIRT3, SIRT4, SIRT5, SIRT6, SIRT7)
exhibit the ability to inhibit HDAC1 (Genbank Accession No. NP_{—}004955, incorporated herein by reference);
exhibit the ability to inhibit HDAC2 (Genbank Accession No. NP_{—}001518, incorporated herein by reference);
exhibit the ability to inhibit HDAC3 (Genbank Accession No. 015739, incorporated herein by reference);
exhibit the ability to inhibit HDAC4 (Genbank Accession No. AAD29046, incorporated herein by reference);
exhibit the ability to inhibit HDAC5 (Genbank Accession No. NP_{—}005465, incorporated herein by reference);
exhibit the ability to inhibit HDAC6 (Genbank Accession No. NP_{—}006035, incorporated herein by reference);
exhibit the ability to inhibit HDAC7 (Genbank Accession No. AAP63491, incorporated herein by reference);
exhibit the ability to inhibit HDAC8 (Genbank Accession No. AAF73428, NM 018486, AF245664, AF230097, each of which is incorporated herein by reference);
exhibit the ability to inhibit HDAC9 (Genbank Accession No. NM 178425, NM 178423, NM 058176, NM 014707, BC111735, NM 058177, each of which is incorporated herein by reference)
exhibit the ability to inhibit HDAC10 (Genbank Accession No. NM 032019, incorporated herein by reference)
exhibit the ability to inhibit HDAC11 (Genbank Accession No. B0009676, incorporated herein by reference);
exhibit the ability to inhibit SIRT1 (Genbank Accession No. NM 003173, NM 001098202, NM 006497, BC 012499, GL 000099, CM000261, each of which is incorporated herein by reference);
exhibit the ability to inhibit SIRT2 (Genbank Accession No. NM 030593, NM 012237, CM000270, AC 000151, NM 033331, CU678487, AK290716, each of which is incorporated herein by reference);
exhibit the ability to inhibit SIRT3 (Genbank Accession No. CM000262, NC 000011, AC 000143, NW 001838015, AC 000054, each of which incorporated herein by reference);
exhibit the ability to inhibit SIRT4 (Genbank Accession No. AM270988, CM000263, NT 166525, NC 000012, NT 009775, AC 000144, each of which is incorporated herein by reference);
exhibit the ability to inhibit SIRT5 (Genbank Accession No. AM270990, AM270988, CM000257, CM000663, GL000052, GL000006, each of which is incorporated herein by reference);
exhibit the ability to inhibit SIRT6 (Genbank Accession No. CM000270, NC 000019, NW 001838477, AC 000151, incorporated herein by reference);
exhibit the ability to inhibit SIRT7 (Genbank Accession No. NC 000017, NT 010663, AC 000149, NW 001838459, each of which is incorporated herein by reference);
exhibit the ability to inhibit tubulin deacetylation (TDAC);
exhibit the ability to inhibit the deacetylation of other acetylated proteins;
exhibit cytotoxic or growth inhibitory effect on cancer cell lines maintained in vitro or in animal studies using a scientifically acceptable cancer cell xenograft model; and/or exhibit a therapeutic profile (e.g., optimum safety and curative effect) that is superior to existing chemotherapeutic agents.

In certain embodiments, the compound's specificity against Class IIa HDACs relative to Class I's inhibition is 1:10. In other embodiments, said specificity is 1:50. In yet other embodiments, said specificity is 1:100. In certain embodiments, said specificity is 1:500. In other embodiments, said specificity is 1:1000.

In certain embodiments, the compound's specificity against Class IIa HDACs relative to Class IIb's inhibition is 1:10. In other embodiments, said specificity is 1:50. In yet other embodiments, said specificity is 1:100. In certain embodiments, said specificity is 1:500. In other embodiments, said specificity is 1:1000.

In certain embodiments, the compound's specificity against Class IIa HDACs relative to Class IV's inhibition is 1:10. In other embodiments, said specificity is 1:50. In yet other embodiments, said specificity is 1:100. In certain embodiments, said specificity is 1:500. In other embodiments, said specificity is 1:1000.

In certain embodiments, the compound's specificity against either HDAC4, 5, 7, 9 relative to either HDAC1, 2, 3, 6, or 8 is 1:10. In certain embodiments, the compound's specificity against either HDAC4, 5, 7, 9 relative to either HDAC1, 2, 3, 6, or 8 is 1:50. In certain embodiments, the compound's specificity against either HDAC4, 5, 7, 9 relative to either HDAC1, 2, 3, 6, or 8 is 1:100. In other embodiments, said specificity is 1:500. In yet other embodiments, said specificity is 1:1000.

As detailed in the exemplification herein, in assays to determine the ability of compounds to inhibit HDAC activity certain inventive compounds exhibit IC₅₀ values ≦100 μM. In certain other embodiments, inventive compounds exhibit IC₅₀ values ≦50 μM. In certain other embodiments, inventive compounds exhibit IC₅₀ values ≦40 μM. In certain other embodiments, inventive compounds exhibit IC₅₀ values ≦30 μM. In certain other embodiments, inventive compounds exhibit IC₅₀ values ≦20 μM. In certain other embodiments, inventive compounds exhibit IC₅₀ values ≦10 μM. In certain other embodiments, inventive compounds exhibit IC₅₀ values ≦7.5 μM. In certain embodiments, inventive compounds exhibit IC₅₀ values ≦5 μM. In certain other embodiments, inventive compounds exhibit IC₅₀ values ≦2.5 μM. In certain embodiments, inventive compounds exhibit IC₅₀ values ≦1 μM. In certain embodiments, inventive compounds exhibit IC₅₀ values ≦0.75 μM. In certain embodiments, inventive compounds exhibit IC₅₀ values ≦0.5 μM. In certain embodiments, inventive compounds exhibit IC₅₀ values ≦0.25 μM. In certain embodiments, inventive compounds exhibit IC₅₀ values ≦0 μM. In certain other embodiments, inventive compounds exhibit IC₅₀ values ≦75 μM. In certain other embodiments, inventive compounds exhibit IC₅₀ values ≦50 μM. In certain other embodiments, inventive compounds exhibit IC₅₀ values ≦25 μM. In certain other embodiments, inventive compounds exhibit IC₅₀ values ≦10 μM. In other embodiments, exemplary compounds exhibit IC₅₀ values ≦7.5 μM. In other embodiments, exemplary compounds exhibit IC₅₀ values ≦5 nM.

In assays to determine the ability of compounds to inhibit cancer cell growth certain inventive compounds exhibit IC₅₀ values ≦100 μM. In certain other embodiments, inventive compounds exhibit IC₅₀ values ≦50 μM. In certain other embodiments, inventive compounds exhibit IC₅₀ values ≦40 μM. In certain other embodiments, inventive compounds exhibit IC₅₀ values ≦30 μM. In certain other embodiments, inventive compounds exhibit IC₅₀ values ≦20 μM. In certain other embodiments, inventive compounds exhibit IC₅₀ values 10 μM. In certain other embodiments, inventive compounds exhibit IC₅₀ values ≦7.5 μM. In certain embodiments, inventive compounds exhibit IC₅₀ values ≦5 μM. In certain other embodiments, inventive compounds exhibit IC₅₀ values ≦2.5 μM. In certain embodiments, inventive compounds exhibit IC₅₀ values ≦1 μM. In certain embodiments, inventive compounds exhibit IC₅₀ values ≦0.75 μM. In certain embodiments, inventive compounds exhibit IC₅₀ values ≦0.5 μM. In certain embodiments, inventive compounds exhibit IC₅₀ values ≦0.25 μM. In certain embodiments, inventive compounds exhibit IC₅₀ values ≦0.1 μM. In certain other embodiments, inventive compounds exhibit IC₅₀ values ≦75 μM. In certain other embodiments, inventive compounds exhibit IC₅₀ values ≦50 μM. In certain other embodiments, inventive compounds exhibit IC₅₀ values ≦25 nM. In certain other embodiments, inventive compounds exhibit IC₅₀ values ≦10 μM. In other embodiments, exemplary compounds exhibit IC₅₀ values ≦7.5 μM. In other embodiments, exemplary compounds exhibit IC₅₀ values ≦5 nM.

### HDAC Assay

The inventive compounds may be tested in any assay for HDAC inhibitor activity. In certain embodiments, the assay for determining the inhibitory effect of an inventive compound on an HDAC protein comprising: incubating the HDAC protein with a substrate of formula:

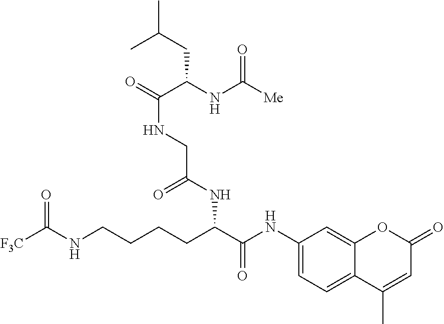 [SMILES: CC(=O)N[C@@H](CC(C)C)C(=O)NCC(=O)N[C@@H](CCCCNC(=O)C(F)(F)F)C(=O)Nc1ccc2c(C)cc(=O)oc2c1]

in the presence of an inventive compound; and determining the activity of the HDAC protein by monitoring the release of 7-amino-4-methylcoumarin after cleavage by trypsin.

In certain embodiments, the assay is carried out at a concentration of the substrate greater than the substrate Kₘ. In other embodiments, the assay is carried out at a concentration of the substrate approximately equivalent to the substrate Kₘ.

In certain embodiments, the HDAC protein is a Class I HDAC. In other embodiments, the HDAC protein is a Class II HDAC. In still other embodiments, the HDAC protein is a Class III HDAC. In further embodiments, the HDAC protein is a Class IV HDAC. In certain embodiments, the HDAC protein is sirtuin. In other embodiments, the HDAC protein is a protein with deacetylase activity.

The assay is suitable for high-throughput screening, and multiple assay may be run in parallel. This aspect of the assay allows for the screening of many test compounds at multiple concentrations at once using more than one HDAC protein.

In certain embodiments, the assay is performed at approximately room temperature. In other embodiments, the assay is performed at approximately 25° C. In still other embodiments, the assay is performed at approximately 37° C. In further embodiments, the assay is performed at approximately 20-40° C. In certain embodiments, the assay is performed below 25° C. In other embodiments, the assay is performed above 25° C. In certain embodiments, the assay is performed at any temperature at which an HDAC enzyme functions. In other embodiments, the assay is performed at a temperature optimum for an HDAC enzyme to function.

In certain embodiments, the assay is performed for approximately 30 seconds to 12 hours. In certain embodiments, the assay is performed for approximately 3 hours. In certain embodiments, the assay is performed for less than 12 hours. In other embodiments, the assay is performed for greater than 12 hours.

In certain embodiments, the assay is performed in water. In other embodiments, the assay is performed in an organic solvent. In still other embodiments, the assay in performed in a buffer. In certain embodiments, the buffer is an assay buffer. In other embodiments, the assay buffer comprises HEPES, KCl, Tween-20, BSA, and TCEP. In further embodiments, the assay buffer is 50 nM HEPES, 100 mM KCl, 0.001% Tween-20, 0.05% BSA, 200 M TCEP, pH 7.4. In certain embodiments, the assay is performed at approximately pH 5.0-6.0. In certain embodiments, the assay is performed at approximately pH 5.0-9.0. In certain embodiments, the assay is performed at a pH optimum for an HDAC enzyme to function.

In certain embodiments, the concentration of the substrate is 1-100 μM.

In certain embodiments, the concentration of the HDAC protein is less than 1 ng/μL. In other embodiments, the concentration of the HDAC protein is greater than 1 ng/μL. In certain embodiments, the concentration of the HDAC protein is less than 5 ng/μL. In other embodiments, the concentration of the HDAC protein is greater than 5 ng/μL. In certain embodiments, the concentration of the HDAC protein is 0.01-5 ng/μL. In other embodiments, the concentration of the HDAC protein is 0.01-0.05 ng/μL. In still other embodiments, the concentration of the HDAC protein is 0.05-0.1 ng/μL. In further embodiments, the concentration of the HDAC protein is 0.1-0.5 ng/μL. In certain embodiments, the concentration of the HDAC protein is 0.5-5 ng/μL.

In certain embodiments, the concentration of HDAC1 is approximately 1-4 ng/μL.

In certain embodiments, the concentration of HDAC2 is approximately 0.5-1.5 ng/μL.

In certain embodiments, the concentration of HDAC3 is approximately 0.1-0.25 ng/μL. In certain embodiments, the concentration of HDAC4 is approximately 0.001-0.025 ng/μL.

In certain embodiments, the concentration of HDAC5 is approximately 0.02-0.04 ng/μL.

In certain embodiments, the concentration of HDAC6 is approximately 0.75-2 ng/μL.

In certain embodiments, the concentration of HDAC7 is approximately 0.001-0.005 ng/μL.

In certain embodiments, the concentration of HDAC8 is approximately 0.02-0.04 ng/p L.

In certain embodiments, the concentration of HDAC9 is approximately 0.02-0.04 ng/μL.

In certain embodiments, the concentration of Sirtuins is approximately 100 to 1500 ng/μL.

In certain embodiments, the assay is performed at the same concentration per test compound. In other embodiments, the assay is performed at multiple concentrations per test compound.

In another aspect, the invention provides an assay for determining the inhibitory effect of a test compound on an HDAC protein comprising: incubating the HDAC protein with a substrate of formula:

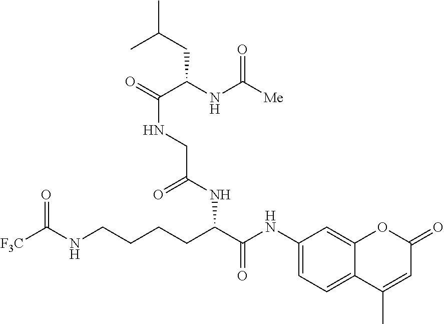 [SMILES: CC(=O)N[C@@H](CC(C)C)C(=O)NCC(=O)N[C@@H](CCCCNC(=O)C(F)(F)F)C(=O)Nc1ccc2c(C)cc(=O)oc2c1]

in the presence of a test compound; and determining the activity of the HDAC protein by monitoring the release of 7-amino-4-methylcoumarin after cleavage by trypsin.

In certain embodiments, the HDAC activity of an inventive compound is measured using assays known to one of ordinary skill in the art, such as assays available in kits from numerous companies (e.g. Biomol, AbCam), or as described by Bedalov et al. (U.S. Pat. No. 7,514,406), incorporated herein by reference.

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance which can be adapted to the practice of this invention in its various embodiments and the equivalents thereof.

These and other aspects of the present invention will be further appreciated upon consideration of the following Examples, which are intended to illustrate certain particular embodiments of the invention but are not intended to limit its scope, as defined by the claims.

### EXAMPLES

The overall lack of potency of hydroxamic acid-based inhibitors for class IIa HDACs is highly unexpected. This observation is based on the available crystal structures of HDAC4 (2VQM) and HDAC7 (3C0Z, 3C10) bound to hydroxamate inhibitors. None of the ligand-protein complexes show the expected bidentate chelation geometry of the central zinc cation, as observed in the structures of ligand-bound human HDAC8 (1T64, 1T69) and bacterial homologs (e.g., 1ZZ1). According to calculations performed by others, the bidentate complexation is a result of the deprotonation of the hydroxamic acid upon ligand binding (Wang, D.-F., Wiest, O., and Helquist, P. (2007) Zinc Binding in HDAC Inhibitors. A DFT Study, J. Org. Chem. 72:5446-5449; incorporated herein by reference). We therefore hypothesized that the active site tyrosine (Tyr298 in HDAC3), which is required for catalytic activity and replaced by a histidine (His843 in HDAC7) in all class IIa HDACs, lowers the pKa of the hydroxamic acid by formation of an hydrogen bond, therefore enabling deprotonation upon binding, which ultimately results in the observed tight binding. This model is also consistent with reports showing that HDAC class IIa His to Tyr mutants not only restore enzymatic activity but also significantly increase the affinity of the gain of function enzymes to hydroxamate based inhibitors (Lahm et al. (2007) Unraveling the hidden catalytic activity of vertebrate class IIa histone deacetylases, Proceedings of the National Academy of Sciences of the United States of America 104:17335-17340; Schuetz et al. (2008) Human HDAC7 harbors a class IIa histone deacetylase-specific zinc binding motif and cryptic deacetylase activity, J. Biol. Chem. 283:11355-11363; Bottomley et al. (2008) Structural and functional analysis of the human HDAC4 catalytic domain reveals a regulatory structural zinc-binding domain, J. Biol. Chem. 283:26694-26704; each of which is incorporated herein by reference).

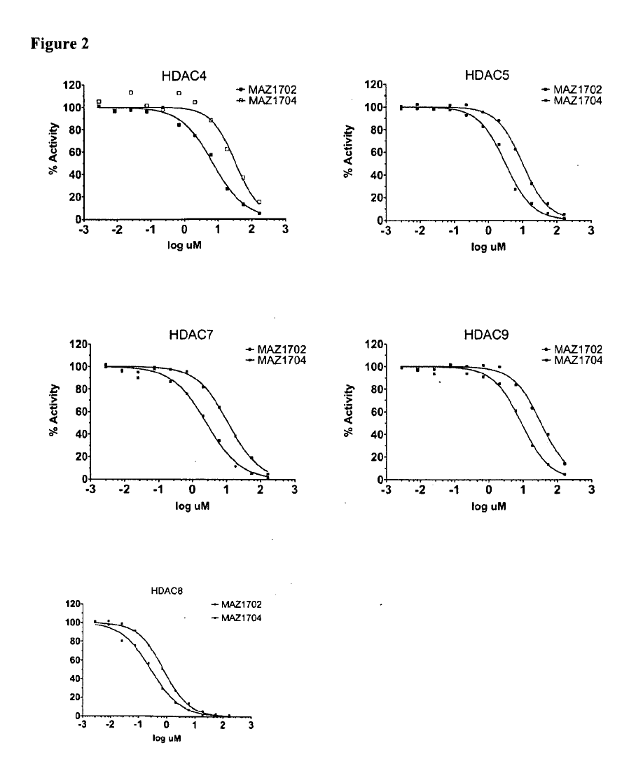

In an effort to probe the hypothesis that a more acidic hydroxamic acid would bind more tightly to class IIa HDACs we synthesized α-fluoro and α,β-difluoro cinnamic hydroxamates. The advantage of lowering the pKa by fluorine substitution over modulating the acidity via substitution of the aromatic system with electron withdrawing groups such as a nitro substituent is two-fold—the additional steric requirements might not be tolerated, and the electron withdrawing effect has to be relayed through the entire n-system significantly altering the overall electronic properties of the ligand. In contrast, substitution with fluorine will only induce a relatively small steric change and will have a direct effect on the neighboring hydroxamic acid group. The pKa of α-fluoro cinnamic hydroxamic acid was determined to be approximately 0.9 units lower than unsubstituted cinammic hydroxamic acid (Dessolin et al., Bull. Soc. Chim. Fr. 2573, (1970); incorporated herein by reference). The fluorinated analog should therefore have significantly increased affinity for class IIa HDACs. The direct comparison of cinnamic hydroxamic acid and α-fluoro cinnamic hydroxamic acid for class IIa enzymes shows a 5-10-fold increase in activity of the fluorinated compounds. Compounds MAZ1702 and MAZ1704 were synthesized. Interestingly, the fluorinated compound binds approximately 3.5-5 fold better to class IIa enzymes (FIG. 2), whereas a 1-1.4-fold increase in activity is observed for HDAC1-3.

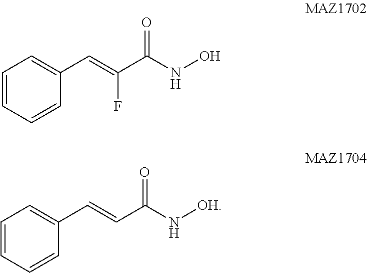 [SMILES: O=C(NO)/C(F)=C/c1ccccc1 ; O=C(/C=C/c1ccccc1)NO]

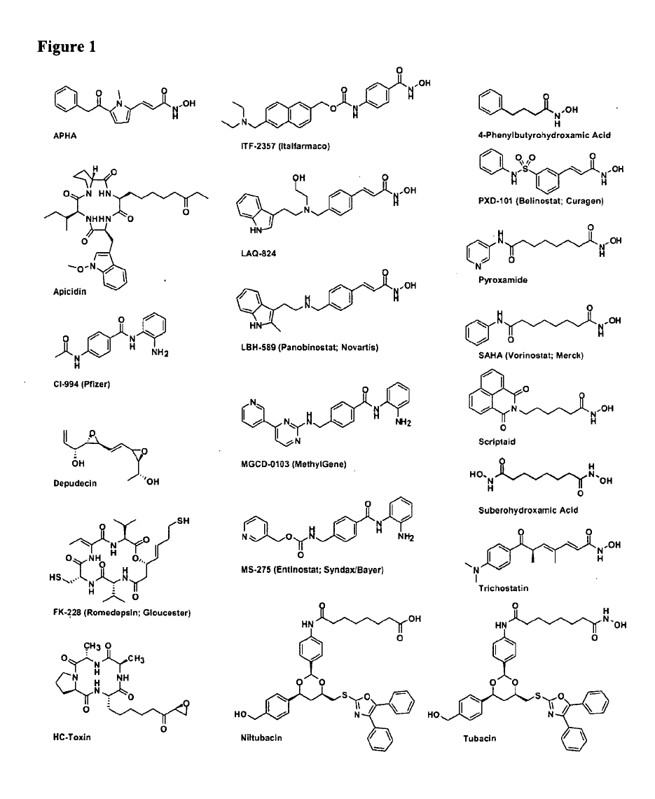

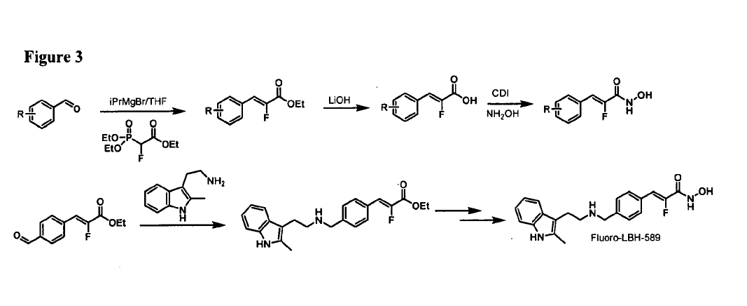

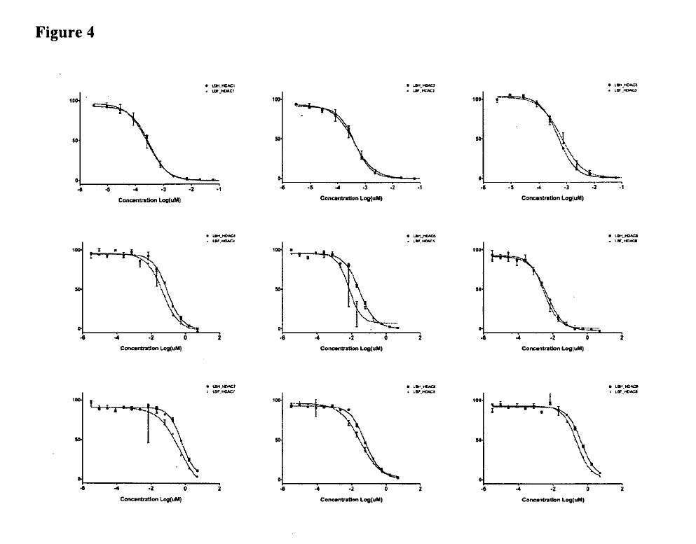

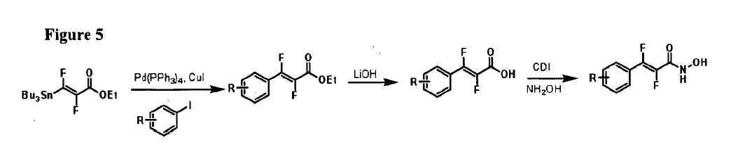

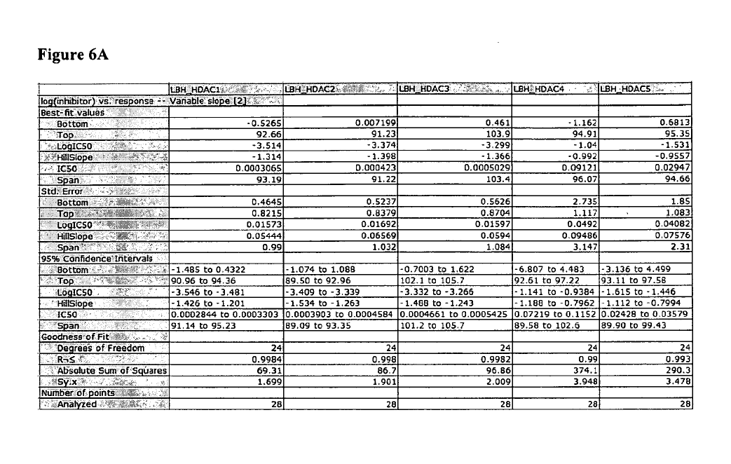

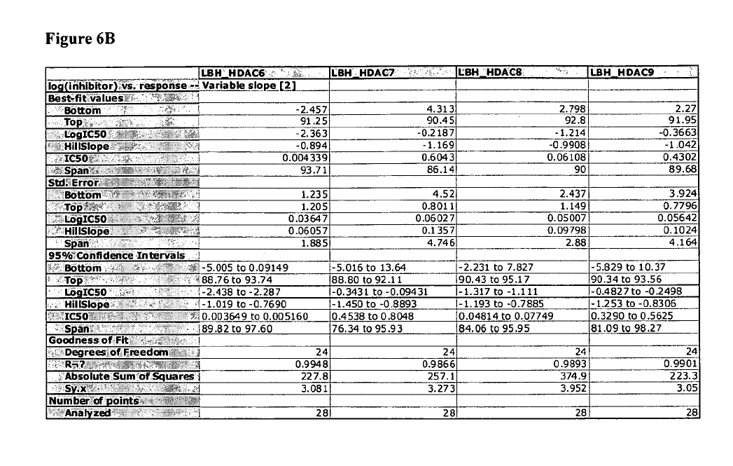

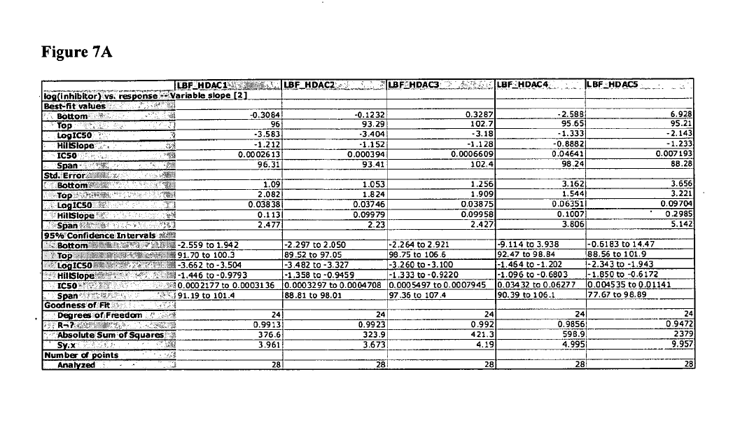

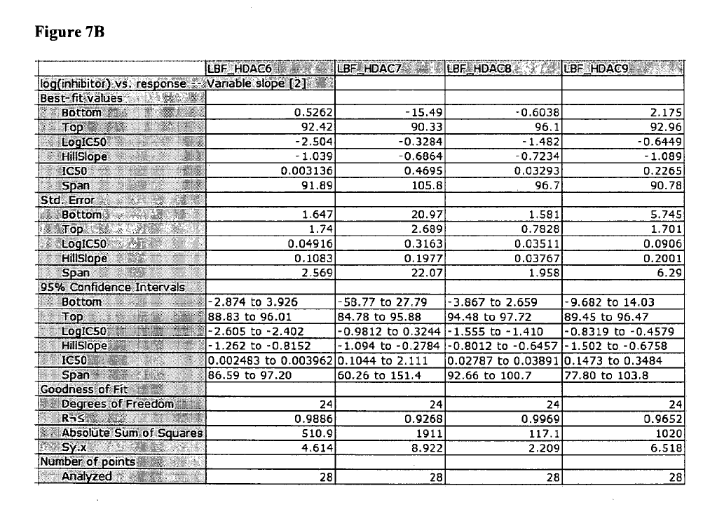

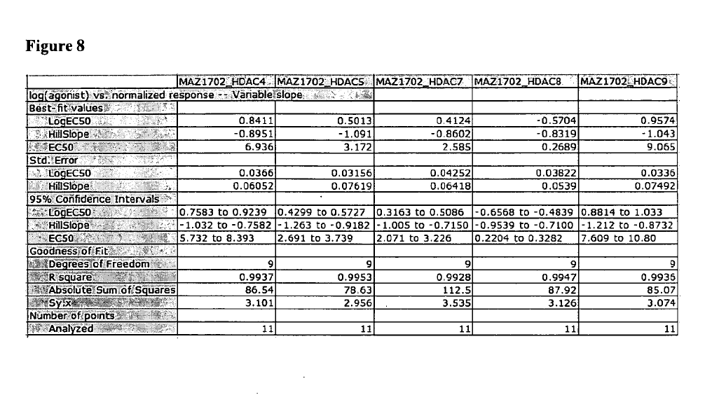

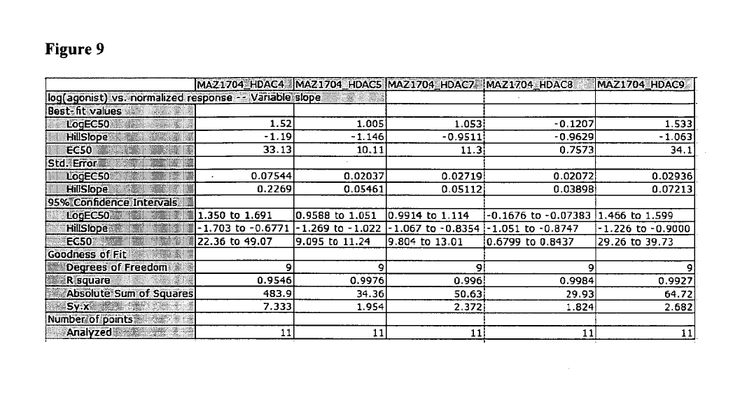

Since LBH-589 and LAQ-824 (as shown in FIG. 1) were identified as two of the few HDAC inhibitors that retained some activity (0.5-5 M range) against class IIa HDACs, an LBH-589 analog with an α-fluoro substituent (LBF, fluoro-LBH-589) was synthesized as illustrated in FIG. 3, adapting the synthetic strategy by Remiszewski et al. The results of profiling the fluorinated analog of LBH-589 (LBF) against human HDACs1-9 are shown in FIG. 4.

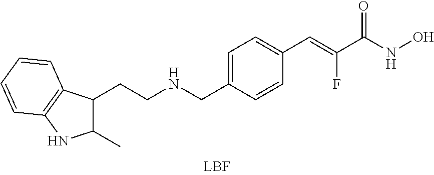 [SMILES: CC1Nc2ccccc2C1CCNCc1ccc(/C=C(\F)C(=O)NO)cc1]

1. A compound of the formula (I): 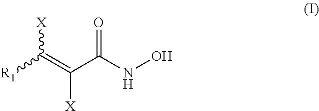
wherein
R₁ is a cyclic or acyclic, substituted or unsubstituted, branched or unbranched aliphatic; a cyclic or acyclic, substituted or unsubstituted, branched or unbranched heteroaliphatic; a substituted or unsubstituted aryl, or a substituted or unsubstituted heteroaryl;
each occurrence of X is independently H, C₁-C₆ alkyl, or F; with the proviso that at least one X is F; or a pharmaceutically acceptable salt thereof.

2. The compound of claim 1, wherein formula (I) is selected from the group consisting of:

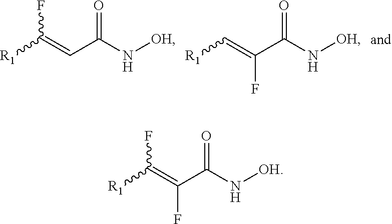

3-15. (canceled)

16. The compound of claim 1, wherein R₁ is selected from the group consisting of

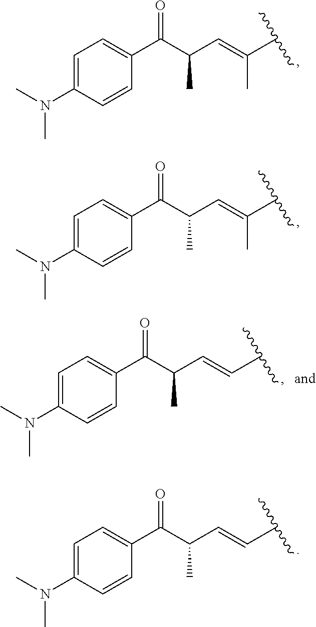 [SMILES: */C(C)=C/[C@@H](C)C(=O)c1ccc(N(C)C)cc1 ; */C(C)=C/[C@H](C)C(=O)c1ccc(N(C)C)cc1 ; */C=C/[C@@H](C)C(=O)c1ccc(N(C)C)cc1 ; */C=C/[C@H](C)C(=O)c1ccc(N(C)C)cc1]

17. The compound of claim 1, wherein R₁ is selected from the group consisting of:

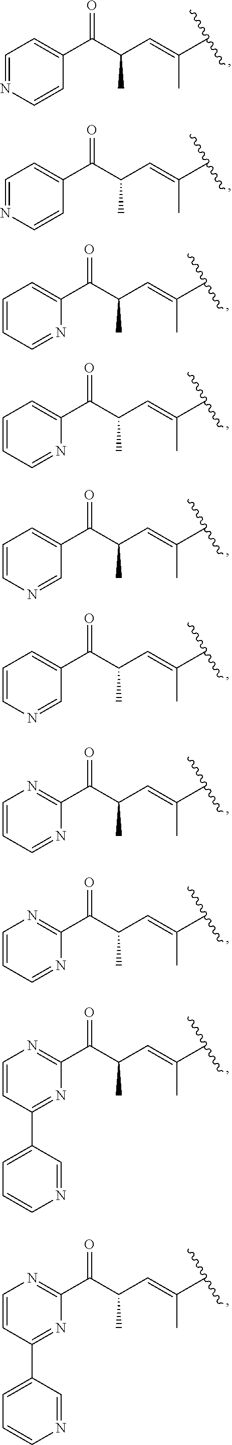

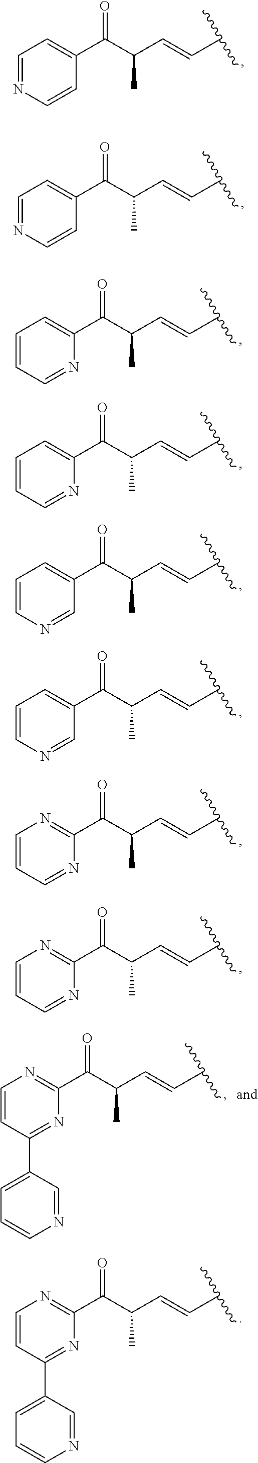

18. The compound of claim 1, wherein R₁ is selected from the group consisting of: 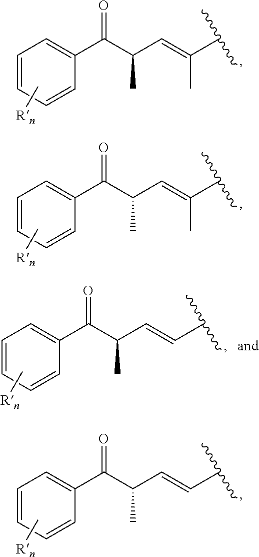
wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branded or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

19. The compound of claim 1, R₁ is substituted or unsubstituted aryl.

20-26. (canceled)

27. The compound of claim 1, wherein R₁ is 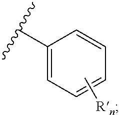
wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

28. (canceled)

29. The compound of claim 27, wherein R₁ is selected from the group consisting of:

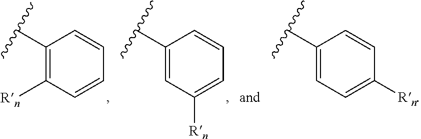

30. The compound of claim 29, wherein R′ is selected from the group consisting of: 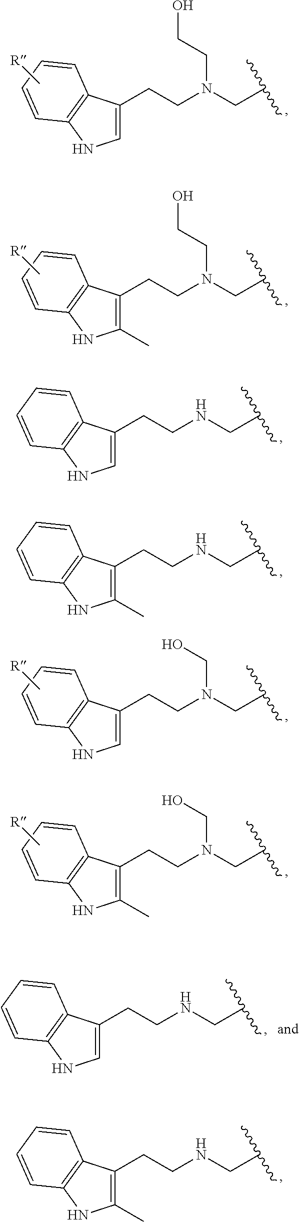
wherein each occurrence of R″ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

31. (canceled)

32. The compound of claim 29, wherein R′ is selected from the group consisting of: 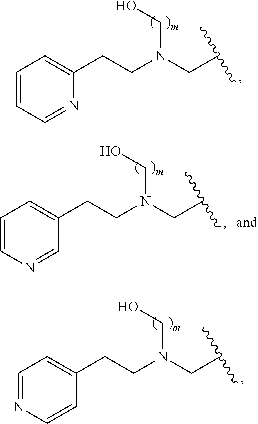
wherein m is an integer between 1 and 6, inclusive.

33. The compound of claim 29, wherein R′ is selected from the group consisting of:

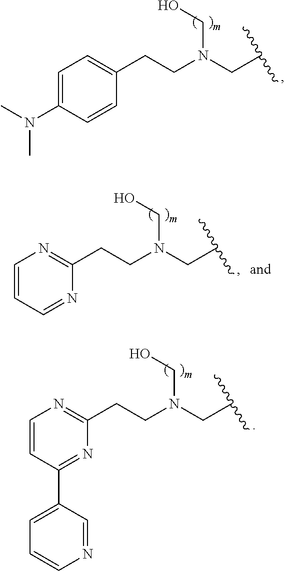

34. The compound of claim 29, wherein R′ is selected from the group consisting of

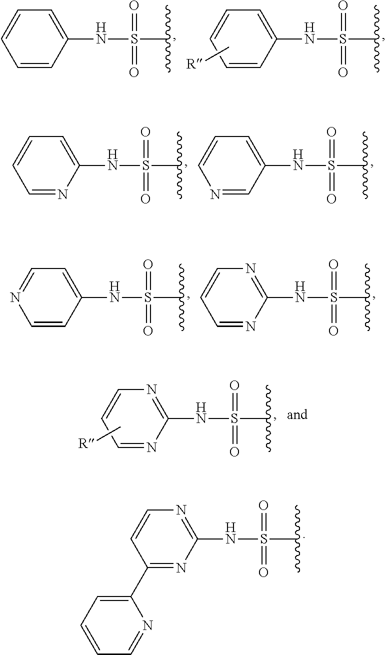

35-49. (canceled)

50. The compound of claim 1, wherein R₁ is selected from the group consisting of:

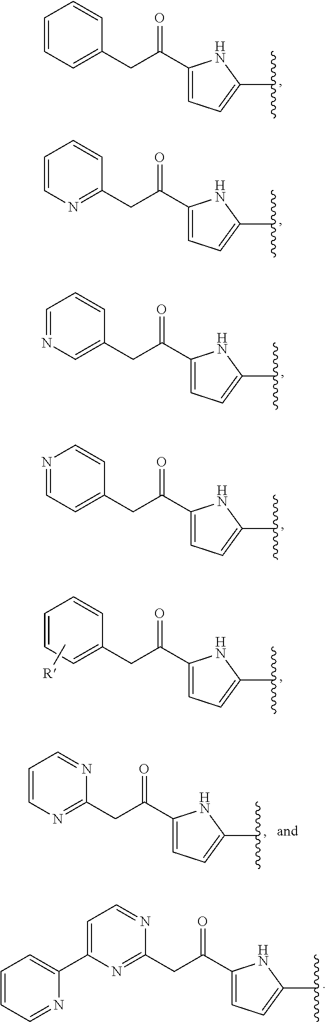

51. (canceled)

52. A compound of the formula (II): 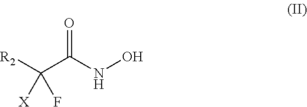
wherein
R₂ is a cyclic or acyclic, substituted or unsubstituted, branched or unbranched aliphatic; a cyclic or acyclic, substituted or unsubstituted, branched or unbranched heteroaliphatic; a substituted or unsubstituted aryl, or a substituted or unsubstituted heteroaryl;
X is independently H, C₁₋₆-alkyl, or F; or a pharmaceutically acceptable salt thereof.

53-88. (canceled)

89. A compound of the formula (III): 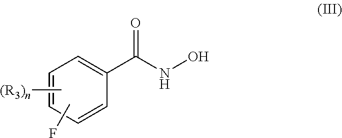
wherein
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy;
n is an integer between 1-4, inclusive; or a pharmaceutically acceptable salt thereof.

90-125. (canceled)

126. The compound of claim 1 of formula: 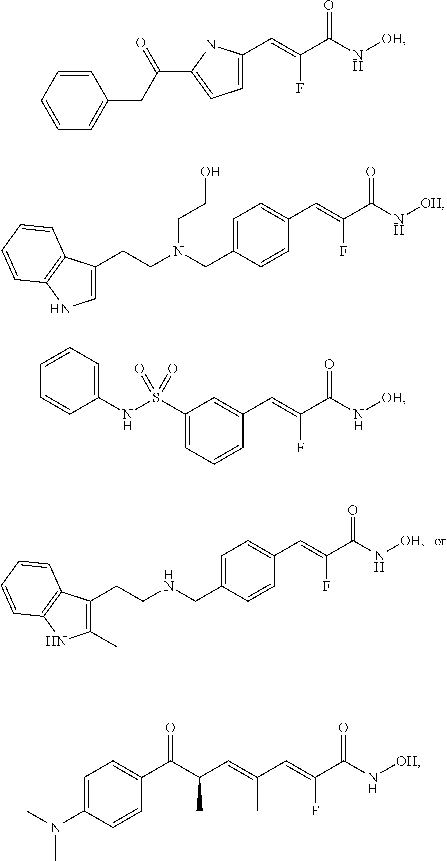
or a pharmaceutically acceptable salt thereof.

127-140. (canceled)

141. A pharmaceutical composition comprising a compound of claim 1, and a pharmaceutically acceptable excipient.

142-147. (canceled)

148. A method of treating a proliferative disease in a subject in need of treatment, comprising administering an effective amount of a composition of claim 141 to the subject.

149-161. (canceled)

162. A method of inhibiting HDAC in a subject, comprising administering an effective amount of a composition of claim 141 to the subject.

163. A method of treating an HDAC associated disease in a subject in need of treatment, comprising inhibiting HDAC in the subject by administering an effective amount of a composition of claim 141 to the subject.
